(19) United States
(12) Patent Application Publication
(10) Pub. No.: US 2006/0069085 A1
(43) Pub. Date: Mar. 30, 2006

(54) Preparation of 4,5-dihydro-pyrazolo[3,4-c]pyrid-2-ones

(76) Inventors: Rulin Zhao, Pennington, NJ (US); Bang-Chi Chen, Plainsboro, NJ (US); Bei Wang, Yardley, PA (US); Huiping Zhang, Belle Mead, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/234,942

(22) Filed: Sep. 26, 2005

Related U.S. Application Data
(60) Provisional application No. 60/613,943, filed on Sep. 28, 2004.

(51) Int. Cl. A61K 31/551; A61K 31/537; A61K 31/506; A61K 31/496; A61K 31/454; A61K 31/4439; A61K 31/4162; C07D 487/02

(52) U.S. Cl. 514/218; 548/360.5; 514/275; 514/254.06; 514/397; 514/405; 540/575; 544/330; 544/366; 546/199; 546/275.7; 514/322; 514/234.2

(57) ABSTRACT

A novel process and intermediates thereof for making 4,5-dihydro-pyrazolo[3,4-c]pyrid-2-ones of the type shown below, as well as the corresponding pyrazoles, from appropriate phenyl hydrazines is described. 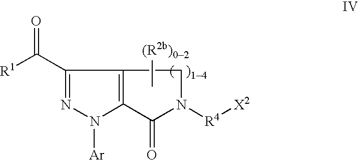 These compounds can be useful as factor Xa inhibitors.

This application claims a benefit of priority from U.S. Provisional Application No. 60/613,943 filed Sep. 28, 2004, the entire disclosure of which is herein incorporated by reference

### FIELD OF THE INVENTION

The present invention relates generally to processes for the preparation of 4,5-dihydro-pyrazolo[3,4-c]pyrid-2-ones, as well as the corresponding pyrazoles, derivatives thereof, and intermediates for the synthesis of the same, such pyrazolo-pyridinones and derivatives being useful as factor Xa inhibitors.

### BACKGROUND OF THE INVENTION

4,5-Dihydro-pyrazolo[3,4-c]pyrid-2-one compounds, like those described in WO 03/26652, are currently being studied as factor Xa inhibitors in clinical settings. Clinical trials and NDA submissions require practical, large-scale synthesis of the active drug and intermediates for making the active drug. Consequently, it is desirable to find new synthetic procedures for making 4,5-dihydro-pyrazolo[3,4-c]pyrid-2-ones.

### SUMMARY OF THE INVENTION

Accordingly, the present invention relates to a novel process for making 4,5-dihydro-pyrazolo[3,4-c]pyrid-2-ones.

The present invention relates to novel intermediates for the syntheses of 4,5-dihydro-pyrazolo[3,4-c]pyrid-2-ones.

These and other embodiments, which will become apparent during the following detailed description of processes relating to 4,5-dihydro-pyrazolo[3,4-c]pyrid-2-ones of formula IV.

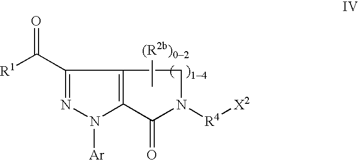

### DETAILED DESCRIPTION OF THE INVENTION

In a first embodiment, the present invention provides a novel process for preparing a compound of formula IV: 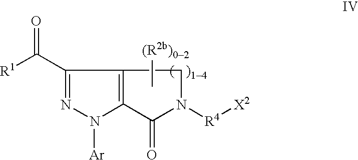
comprising:

(a) contacting a compound of formula I with a compound of formula II to form a compound of formula III;

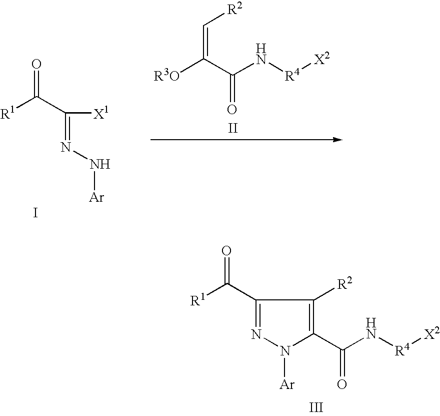

(b) cyclizing a compound of formula III to a compound of formula IV; wherein:

X¹ is a leaving group selected from Cl, Br, and I;

X² is a leaving group selected from Cl, Br, I, OSO₂Me, OSO₂CF₃, OSO₂Ph, and OSO₂Ph-p-Me;

R¹ is selected from C₁₋₆ alkyl, C₀₋₆ alkylene-phenyl, O—C₁₋₆ alkyl, and O—C₀₋₆ alkylene-phenyl;

R² is C₁₋₄ alkylene-R^{2a}, wherein the alkylene portion of R² is substituted with 0-2 R^{2b};

R^{2a} is OH;

R^{2b} is selected from C₁₋₄ alkyl, phenyl, and benzyl;

R³ is selected from C₁₋₆, alkyl, phenyl, and benzyl;

alternatively, in formula II, R³O—*C═CH—R² forms a group selected from:

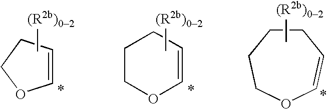

wherein * indicates the point of attachment to the remainder of formula II, provided that when R³O—*C═CH—R² forms a ring, then R² in formula III is C₂₋₄ alkylene-OH and the alkylene portion is substituted with 0-2 R^{2b};

R⁴ is a 5-10 membered aromatic carbocyclic or heterocyclic ring consisting of carbon atoms and 0-4 heteroatoms selected from O, S(O)ₚ, and N and R⁴ is substituted with 0-2 groups selected from F and C₁₋₄ alkyl;

Ar is

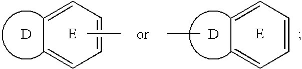

ring D, including the two atoms of Ring E to which it is attached, is a 5-6 membered ring consisting of: carbon atoms and 0-2 heteroatoms selected from the group consisting of N, O, and S(O)ₚ;

ring D is substituted with 0-2 R and there are 0-3 ring double bonds;

E is selected from phenyl, pyridyl, pyrimidyl, pyrazinyl, and pyridazinyl, and is substituted with 1-2 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-2 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-1 carbonyl and 1-2 R and there are 0-3 ring double bonds;

R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NHC(═NR⁸)NR⁷R⁹, ONHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;

alternatively, when 2 R groups are attached to adjacent atoms, they combine to form methylenedioxy or ethylenedioxy;

R⁵, at each occurrence, is selected from CF₃, OH, C₁₋₄ alkoxy, C₁₋₆ alkyl, —(CH₂)ᵣ-C₃₋₁₀ carbocycle substituted with 0-2 R^{5a}, and —(CH₂)ᵣ-5-10 membered heterocycle containing from 1-4 heteroatoms selected from the group consisting of N, O, and S(O)ₚ, and substituted with 0-2 R^{5a};

R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣ-C(═NR⁶)NR⁶R^{6a}, (CH₂)ᵣNR⁶C(═NR⁶)NR⁶R^{6a}, (CH₂)ᵣSO₂NR⁶R^{6a}, (CH₂)ᵣNR⁶SO₂NR⁶R^{6a}, (CH₂)ᵣNR⁶SO₂—C₁₋₄ alkyl, (CH₂)ᵣNR⁶SO₂CF₃, (CH₂)ᵣNR⁶SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;

R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

alternatively, NR⁶R^{6a} forms a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;

R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;

R⁸, at each occurrence, is selected from H, C₁₋₆ alkyl, and (CH₂)ₙ-phenyl;

alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;

R⁹, at each occurrence, is selected from H, C₁₋₆ alkyl, and (CH₂)ₙ-phenyl;

alternatively, R⁴—X² is selected from:

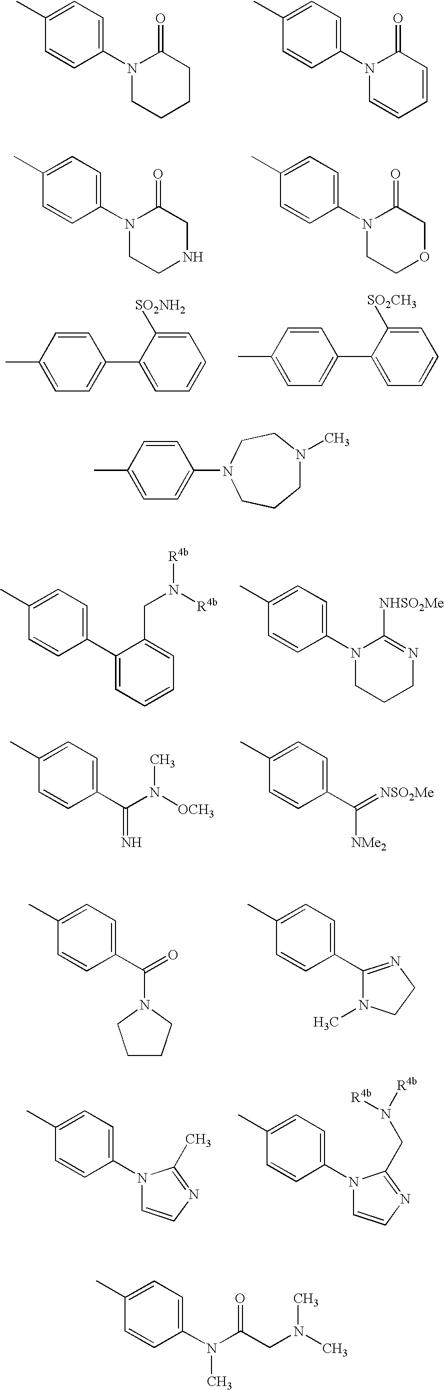

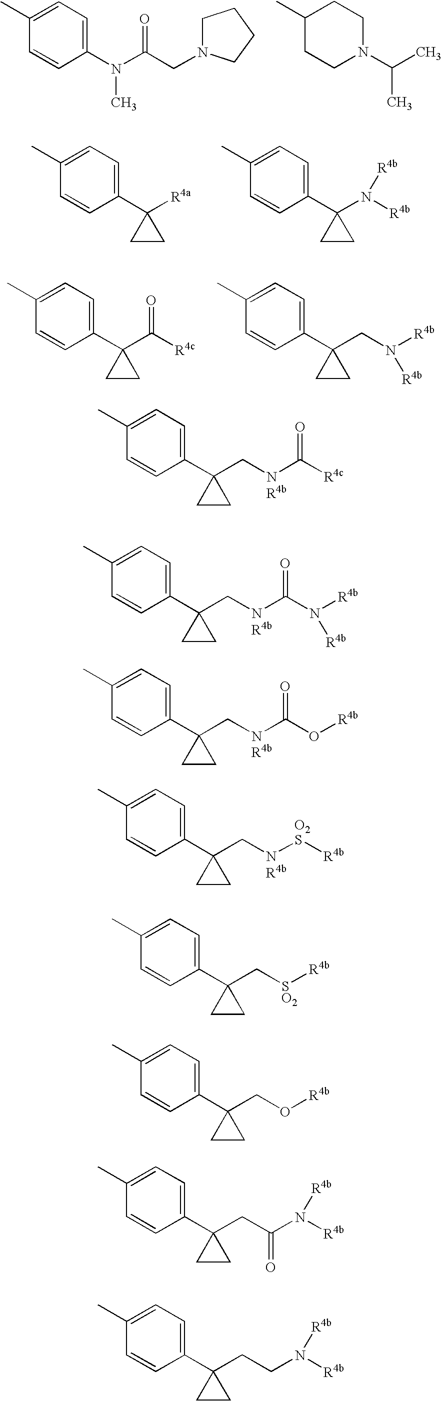

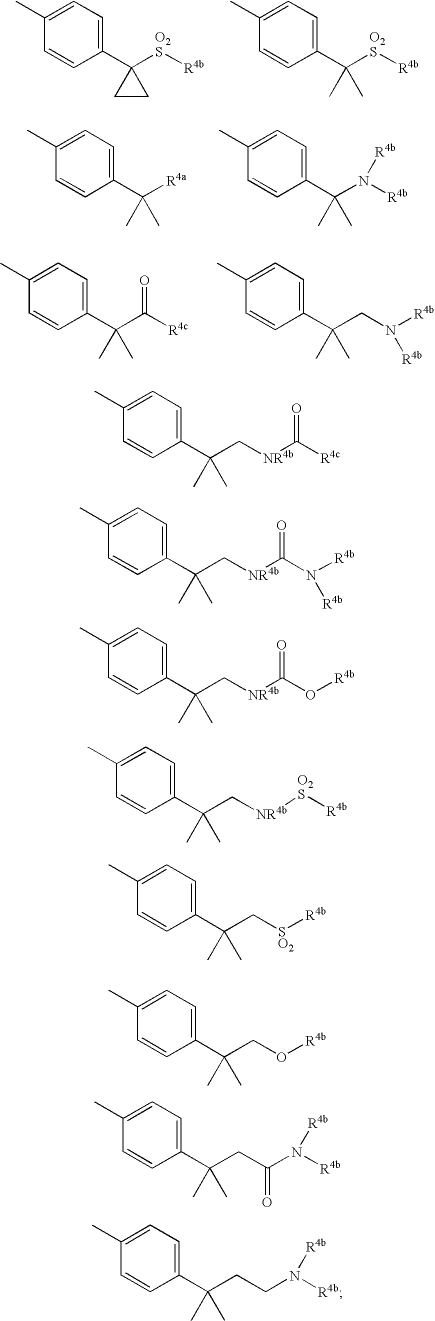

R^{4a} is substituted with 0-2 R^{4d} and selected from morpholine, 1,1-dioxo-thiomorpholine, dihydropyridine, piperidine, piperazine, pyrrolidine, imidazole, imidazoline, imidazolidine, oxazoline, and thiazoline;

R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;

R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;

R^{4d} is selected from ═O, OH, OCH₃, and CH₃;

n, at each occurrence, is selected from 0, 1, 2, and 3;

p, at each occurrence, is selected from 0, 1, and 2;

r, at each occurrence, is selected from 0, 1, 2, 3, 4, 5, and 6; and

t, at each occurrence, is selected from 0, 1, 2, and 3.

In a second embodiment, the present invention provides a novel process for preparing a compound of formula IV, comprising:

(a₁) contacting a compound of formula I with a compound of formula II in the presence of a first base and a first solvent to form a cycloaddition product;

(a₂) contacting the cycloaddition product from reaction (a₁) with a first acid to form a compound of formula III;

(b) cyclizing a compound of formula III to a compound of formula IV through one of reaction sequences (b₁) or (b₂);

(b₁) converting R^{2a} of formula III to leaving group X³, followed by contacting the resulting product with a second base in the presence of a second solvent;

(b₂) alternatively, contacting a compound of formula III with a phosphine reagent and a diazo reagent under water removing conditions;

wherein:

X³ is a leaving group selected from Cl, Br, I, OSO₂Me, OSO₂CF₃, OSO₂Ph, and OSO₂Ph-p-Me;

the first base is selected from a tertiary amine base and a pyridine base;

the first acid is selected from HCl, AcOH, H₂SO₄, and H₃PO₄;

the first solvent is an aprotic solvent;

the second base is an alkoxide; and

the second solvent is selected from an alcoholic solvent and an aprotic solvent.

In a third embodiment, the present invention provides a novel process for preparing a compound of formula IVa: 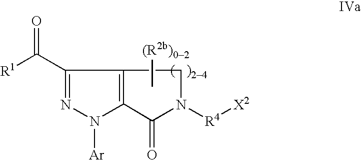
comprising:

(a₁) contacting a compound of formula Ia with a compound of formula IIa in the presence of a first base and a first solvent to form a cycloaddition product;

(a₂) contacting the cycloaddition product from reaction (a₁) with a first acid to form a compound of formula IIIa;

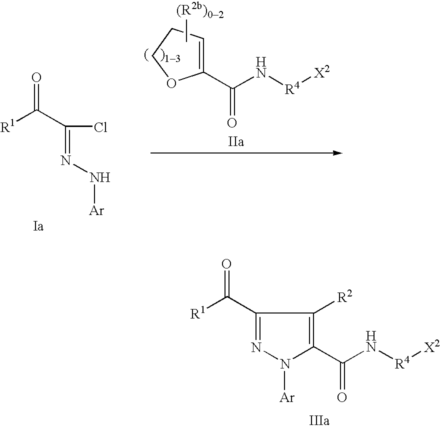

(b) cyclizing a compound of formula IIIa to a compound of formula IVa through one of reaction sequences (b₁) or (b₂);

(b₁) converting the OH group of R² in formula IIIa to leaving group X³ and contacting the resulting product with a second base in the presence of a second solvent;

(b₂) alternatively, contacting a compound of formula IIIa with a phosphine reagent and a diazo reagent under water removing conditions; wherein:

the first base is triethylamine;

the first solvent is selected from toluene and ethyl acetate;

the first acid is HCl;

the second base is a C₁₋₆ alkoxide and the counterion is selected from Li, Na, K, Li, and Mg;

the second solvent is selected from C₁₋₆ alcohol, DMF, and DMSO;

X² is a leaving group selected from Br and I;

R¹ is selected from O—C₁₋₆ alkyl and O—C₀₋₆ alkylene-phenyl;

R² is selected from C₂₋₄ alkylene-OH, wherein the alkylene portion of R² is substituted with 0-2 R^{2b};

R^{2b} is selected from C₁₋₄ alkyl, phenyl, and benzyl;

X³ is a leaving group selected from OSO₂Me, OSO₂CF₃, OSO₂Ph, and OSO₂Ph-p-Me;

R⁴ is a 5-6 membered aromatic carbocyclic or heterocyclic ring consisting of carbon atoms and 0-4 heteroatoms selected from O, S(O)ₚ, and N;

Ar is

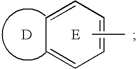

ring D, including the two atoms of Ring E to which it is attached, is a 5-6 membered ring consisting of: carbon atoms and 0-2 heteroatoms selected from the group consisting of N, O, and S(O)ₚ;

ring D is substituted with 0-2 R and there are 0-3 ring double bonds;

E is selected from phenyl and pyridyl and is substituted with 1-2 R;

alternatively, ring D is absent and ring E is selected from phenyl, pyridyl, and thienyl, and ring E is substituted with 1-2 R;

R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;

R⁵, at each occurrence, is selected from CF₃, OH, C₁₋₄ alkoxy, C₁₋₆ alkyl, —(CH₂)ᵣ—C₅₋₆ carbocycle substituted with 0-2 R^{5a}, and —(CH₂)ᵣ-5-6 membered heterocycle containing from 1-4 heteroatoms selected from the group consisting of N, O, and S(O)ₚ, and substituted with 0-2 R^{5a};

R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣSO₂NR⁶R^{6a}, CH₂)ᵣS(O)ₚ—C₁₋₄ alkyl, (CH₂)ᵣS(O)ₚ-phenyl, and (CH₂)ᵣ(CF₂)ᵣCF₃;

R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

alternatively, NR⁶R^{6a} form a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;

R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;

R⁸, at each occurrence, is selected from H, C₁₋₆ alkyl, and (CH₂)ₙ-phenyl;

alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;

R⁹, at each occurrence, is selected from H, C₁₋₆ alkyl, and (CH₂)ₙ-phenyl;

alternatively, R⁴—X² is selected from:

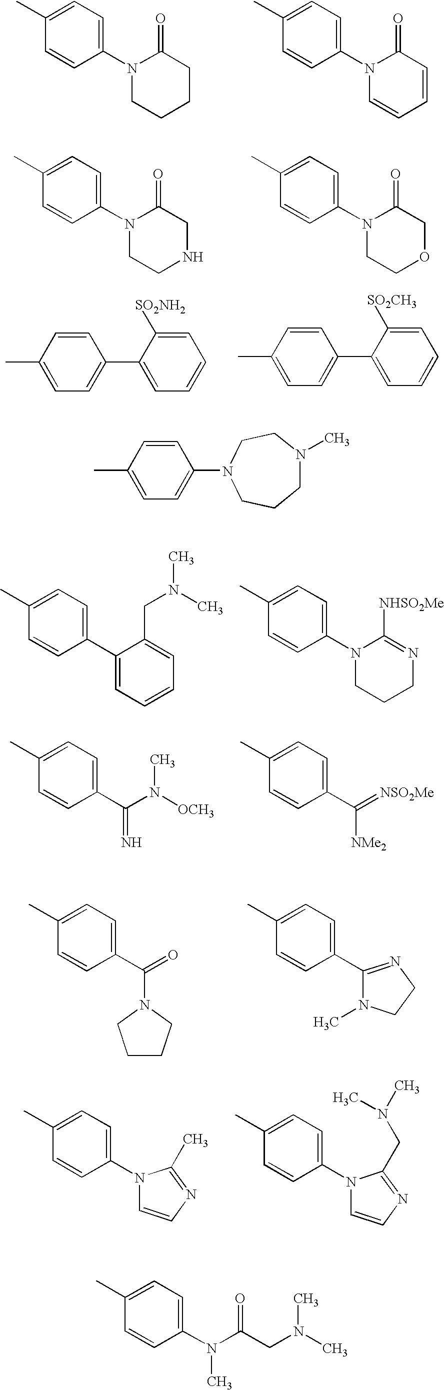

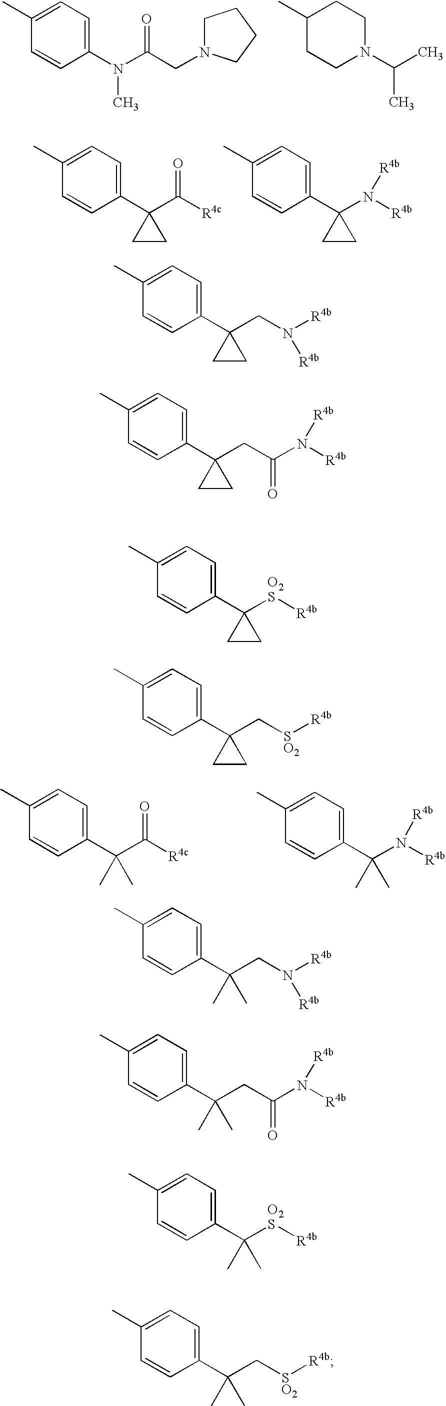

R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;

R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;

R^{4d} is selected from ═O, OH, OCH₃, and CH₃;

n, at each occurrence, is selected from 0, 1, 2, and 3;

p, at each occurrence, is selected from 0, 1, and 2;

r, at each occurrence, is selected from 0, 1, 2, and 3; and

t, at each occurrence, is selected from 0, 1, 2, and 3.

In a fourth embodiment, the present invention provides a novel process for preparing a compound of formula IVb: 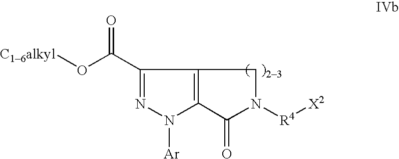
comprising:

(a₁) contacting a compound of formula Ib with a compound of formula IIb in the presence of a first base and a first solvent to form a cycloaddition product;

(a₂) contacting the cycloaddition product from reaction (a₁) with a first acid to form a compound of formula IIIb;

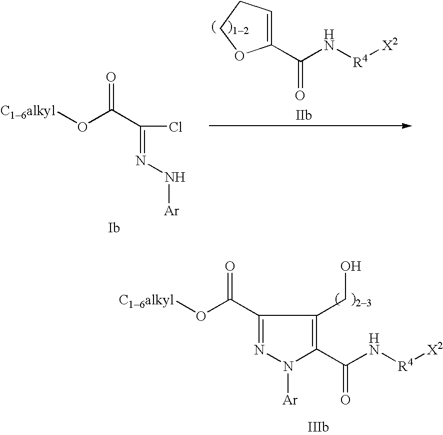

(b₂) converting a compound of formula IIIb to formula IIIb₁ and contacting the compound of formula IIIb₁ with a second base in the presence of a second solvent to form a compound of formula IVb; 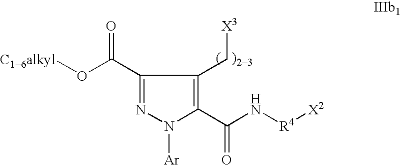
wherein:

the first base is triethylamine;

the first solvent is ethyl acetate

the first acid is HCl;

the second base is NaOEt;

the second solvent is EtOH;

X² is I;

X³ is a leaving group selected from OSO₂Me and OSO₂Ph-p-Me;

R⁴ is selected from phenyl, pyridyl, and pyrimidyl;

Ar is selected from phenyl, 2-fluorophenyl, 3-aminomethyl-phenyl, 3-amidino-phenyl, 3-amido-phenyl, 3-chlorophenyl, 4-methoxyphenyl, 2-naphthyl, 1-fluoro-2-naphthyl, 1-cyano-2-naphthyl, and 6-chloro-2-naphthyl; and

p, at each occurrence, is selected from 0, 1, and 2.

In a fifth embodiment, the present invention provides a novel process for preparing a compound of formula IVc: 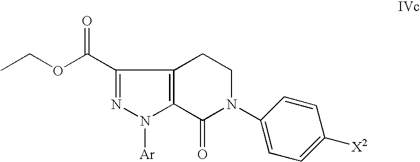
comprising:

(a₁) contacting a compound of formula Ic with a compound of formula IIc in the presence of a triethylamine and a ethyl acetate to form a cycloaddition product;

(a₂) contacting the cycloaddition product from reaction (a₁) with a HCl to form a compound of formula IIIc;

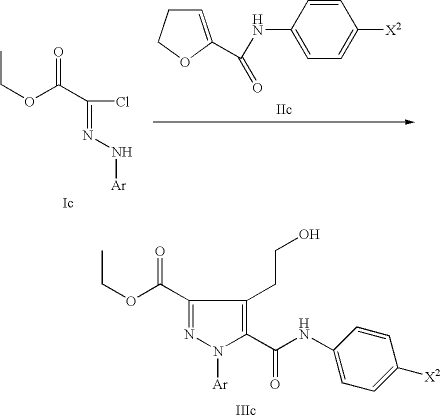

(b₁) converting a compound of formula IIIc to formula IIIc₁ and contacting the compound of formula IIIc₁ with a NaOEt in the presence of a EtOH to form a compound of formula IVc; 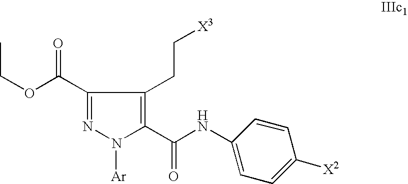
wherein:

X² is I;

X³ is OSO₂Me; and

Ar is selected from phenyl, 2-fluorophenyl, 3-chlorophenyl, and 4-methoxyphenyl.

In a sixth embodiment, the present invention provides a novel process for preparing a compound of formula IVc, wherein the compound of formula IIIc is converted to the compound of formula IIIc_{1A} by contacting formula IIIc with mesyl chloride in the presence of a third base and a third solvent; 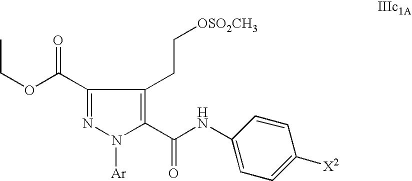
wherein:

the third base is a tertiary amine base; and

the third solvent is an aprotic solvent.

In a seventh embodiment, the present invention provides a novel process, wherein:

the third base is a triethylamine; and

the third solvent is dichloromethane.

In an eighth embodiment, the present invention provides a novel process for preparing a compound of formula VI: 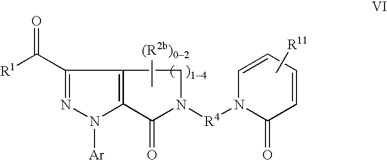
comprising:

(c) contacting a compound of formula IV with a compound of formula V in the presence of a metal salt and a fourth solvent; 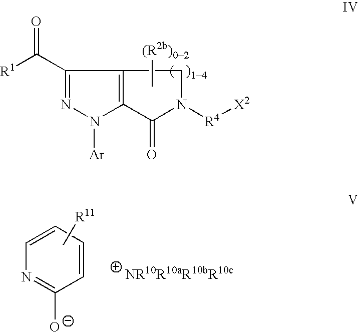
wherein:

metal salt is selected from a copper and a palladium salt;

the fourth solvent is an alcoholic or an aprotic solvent;

X² is a leaving group selected from Cl, Br, I, OSO₂Me, OSO₂CF₃, OSO₂Ph, and OSO₂Ph-p-Me;

R¹ is selected from C₁₋₆ alkyl, C₀₋₆ alkylene-phenyl, O—C₁₋₆ alkyl, and O—C₀₋₆ alkylene-phenyl;

R^{2b} is selected from C₁₋₄ alkyl, phenyl, and benzyl;

R⁴ is a 5-10 membered aromatic carbocyclic or heterocyclic ring consisting of carbon atoms and 0-4 heteroatoms selected from O, S(O)ₚ, and N and R⁴ is substituted with 0-2 groups selected from F and C₁₋₄ alkyl;

Ar is

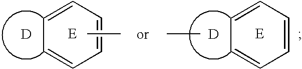

ring D, including the two atoms of Ring E to which it is attached, is a 5-6 membered ring consisting of: carbon atoms and 0-2 heteroatoms selected from the group consisting of N, O, and S(O)ₚ;

ring D is substituted with 0-2 R and there are 0-3 ring double bonds;

E is selected from phenyl, pyridyl, pyrimidyl, pyrazinyl, and pyridazinyl, and is substituted with 1-2 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-2 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-1 carbonyl and 1-2 R and there are 0-3 ring double bonds;

R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NHC(═NR⁸)NR⁷R⁹, ONHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;

alternatively, when 2 R groups are attached to adjacent atoms, they combine to form methylenedioxy or ethylenedioxy;

R⁵, at each occurrence, is selected from CF₃, OH, C₁₋₄ alkoxy, C₁₋₆ alkyl, —(CH₂)ᵣ—C₃₋₁₀ carbocycle substituted with 0-2 R^{5a}, and —(CH₂)ᵣ-5-10 membered heterocycle containing from 1-4 heteroatoms selected from the group consisting of N, O, and S(O)ₚ, and substituted with 0-2 R^{5a};

R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣ—C(═NR⁶)NR⁶R^{6a}, (CH₂)ᵣNR⁶C(═NR⁶)NR⁶R^{6a}, (CH₂)ᵣSO₂NR⁶R^{6a}, (CH₂)ᵣNR⁶SO₂NR⁶R^{6a}, (CH₂)ᵣNR⁶SO₂—C₁₋₄ alkyl, (CH₂)ᵣNR⁶SO₂CF₃, (CH₂)ᵣNR⁶SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;

R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

alternatively, NR⁶R^{6a} form a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;

R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;

R⁸, at each occurrence, is selected from H, C₁₋₆ alkyl, and (CH₂)ₙ-phenyl;

alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;

R⁹, at each occurrence, is selected from H, C₁₋₆ alkyl, and (CH₂)ₙ-phenyl;

R¹⁰ is selected from C₁₋₂₀ alkyl, phenyl, and benzyl;

R^{10a} is selected from C₁₋₁₈ alkyl, phenyl, and benzyl;

R^{10c} is selected from C₁₋₁₈ alkyl, phenyl, and benzyl;

R^{10c} is selected from C₁₋₁₈ alkyl, phenyl, and benzyl;

R¹¹ is selected from H, C₁₋₄ alkyl, OC₁₋₄, alkyl, F, Br, Cl, CN, NO₂, phenyl, and benzyl;

n, at each occurrence, is selected from 0, 1, 2, and 3;

p, at each occurrence, is selected from 0, 1, and 2;

r, at each occurrence, is selected from 0, 1, 2, 3, 4, 5, and 6; and

t, at each occurrence, is selected from 0, 1, 2, and 3.

In a ninth embodiment, the present invention provides a novel process for preparing a compound of formula VIa: 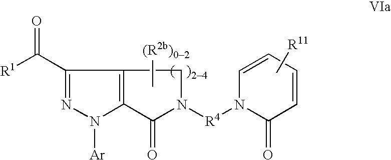
comprising:

(c) contacting a compound of formula IVa with a compound of formula V in the presence of a metal salt and a fourth solvent;

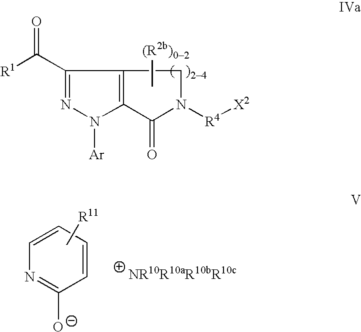

wherein:

metal salt is a copper (I) salt;

the fourth solvent is an aprotic solvent;

X² is a leaving group selected from Br and I;

R¹ is selected from O—C₁₋₆ alkyl and O—C₀₋₆ alkylene-phenyl;

R^{2b} is selected from C₁₋₄ alkyl, phenyl, and benzyl;

R⁴ is a 5-6 membered aromatic carbocyclic or heterocyclic ring consisting of carbon atoms and 0-4 heteroatoms selected from O, S(O)ₚ, and N;

Ar is

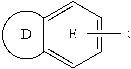

ring D, including the two atoms of Ring E to which it is attached, is a 5-6 membered ring consisting of: carbon atoms and 0-2 heteroatoms selected from the group consisting of N, O, and S(O)ₚ;

ring D is substituted with 0-2 R and there are 0-3 ring double bonds;

E is selected from phenyl and pyridyl and is substituted with 1-2 R;

alternatively, ring D is absent and ring E is selected from phenyl, pyridyl, and thienyl, and ring E is substituted with 1-2 R;

R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;

R⁵, at each occurrence, is selected from CF₃, OH, C₁₋₄ alkoxy, C₁₋₆ alkyl, —(CH₂)ᵣ—C₅₋₆ carbocycle substituted with 0-2 R^{5a}, and —(CH₂)ᵣ-5-6 membered heterocycle containing from 1-4 heteroatoms selected from the group consisting of N, O, and S(O)ₚ, and substituted with 0-2 R^{5a};

R^{5a}, at each occurrence, is selected from H, —O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣSO₂NR⁶R^{6a}, CH₂)ᵣS(O)ₚ—C₁₋₄ alkyl, (CH₂)ᵣS(O)ₚ-phenyl, and (CH₂)ᵣ(CF₂)ᵣCF₃;

R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

alternatively, NR⁶R^{6a} form a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;

R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;

R⁸, at each occurrence, is selected from H, C₁₋₆ alkyl, and (CH₂)ₙ-phenyl;

alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;

R⁹, at each occurrence, is selected from H, C₁₋₆ alkyl, and (CH₂)ₙ-phenyl;

R¹⁰ is selected from C₁₋₆ alkyl, phenyl, and benzyl;

R^{10a} is selected from C₁₋₆ alkyl, phenyl, and benzyl;

R^{10c} is selected from C₁₋₆ alkyl, phenyl, and benzyl;

R^{10c} is selected from C₁₋₆ alkyl, phenyl, and benzyl;

R¹¹ is selected from H, C₁₋₄ alkyl, OC₁₋₄ alkyl, F, Br, Cl, and benzyl;

n, at each occurrence, is selected from 0, 1, 2, and 3;

p, at each occurrence, is selected from 0, 1, and 2;

r, at each occurrence, is selected from 0, 1, 2, and 3; and

t, at each occurrence, is selected from 0, 1, 2, and 3.

In a tenth embodiment, the present invention provides a novel process for preparing a compound of formula VIb: 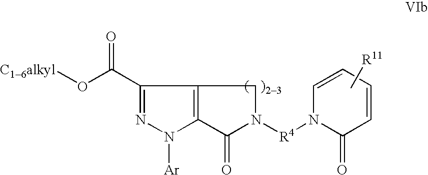
comprising:

(c) contacting a compound of formula IVb with a compound of formula V in the presence of a metal salt and a fourth solvent; 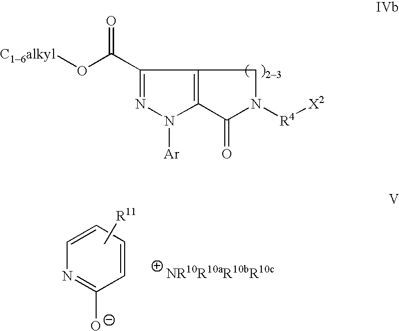
wherein:

metal salt is selected from CuI and CuOTf;

the fourth solvent is DMF;

X² is I;

R⁴ is a 6 membered aromatic carbocyclic or heterocyclic ring consisting of carbon atoms and 0-2 N atoms;

Ar is selected from phenyl, 2-fluorophenyl, 3-aminomethyl-phenyl, 3-amidino-phenyl, 3-amido-phenyl, 3-chlorophenyl, 4-methoxyphenyl, 2-naphthyl, 1-fluoro-2-naphthyl, 1-cyano-2-naphthyl, and 6-chloro-2-naphthyl;

R¹⁰ is selected from C₁₋₆ alkyl;

R^{10a} is selected from C₁₋₆ alkyl;

R^{10c} is selected from C₁₋₆ alkyl;

R^{10c} is selected from C₁₋₆ alkyl;

R¹¹ is H; and

p, at each occurrence, is selected from 0, 1, and 2.

In an eleventh embodiment, the present invention provides a novel process for preparing a compound of formula VIc: 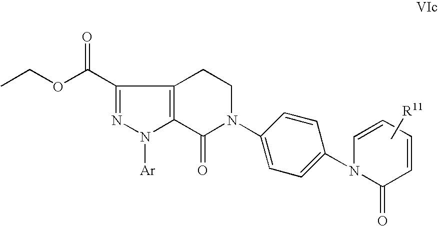
comprising:

(c) contacting a compound of formula IVc with a compound of formula V in the presence of a CuI and a DMF; 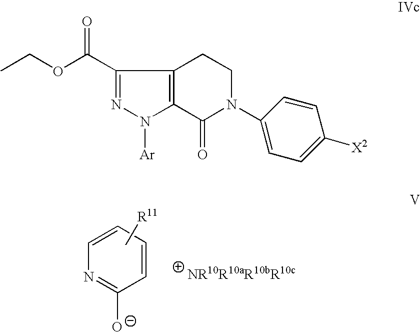
wherein:

X² is I;

Ar is selected from phenyl, 2-fluorophenyl, 3-chlorophenyl, and 4-methoxyphenyl;

R is selected from H, F, Cl, and OCH₃;

R¹⁰ is n-butyl;

R^{10a} is n-butyl;

R^{10c} is n-butyl;

R^{10c} is n-butyl; and

R¹¹ is H.

In a twelfth embodiment, the present invention provides a novel process for preparing a compound of formula IIId, comprising:

(a) contacting a compound of formula Id with a compound of formula IId to form a compound of formula IIId; 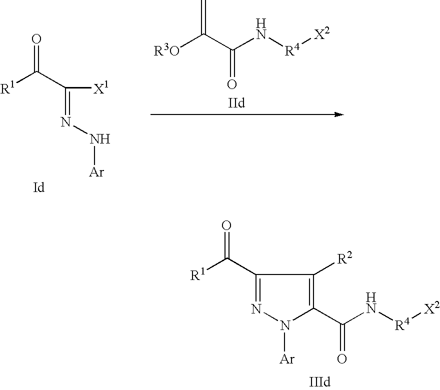
wherein:

X¹ is a leaving group selected from Cl, Br, and I;

X² is a leaving group selected from Cl, Br, I, OSO₂Me, OSO₂CF₃, OSO₂Ph, and OSO₂Ph-p-Me;

R¹ is selected from C₁₋₆ alkyl, C₀₋₆ alkylene-phenyl, O—C₁₋₆ alkyl, and O—C₀₋₆ alkylene-phenyl;

R³ is selected from C₁₋₆ alkyl, phenyl, and benzyl;

R⁴ is a 5-10 membered aromatic carbocyclic or heterocyclic ring consisting of carbon atoms and 0-4 heteroatoms selected from O, S(O)ₚ, and N and R⁴ is substituted with 0-2 groups selected from F and C₁₋₄ alkyl;

Ar is

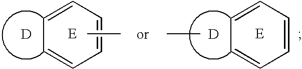

ring D, including the two atoms of Ring E to which it is attached, is a 5-6 membered ring consisting of: carbon atoms and 0-2 heteroatoms selected from the group consisting of N, O, and S(O)ₚ;

ring D is substituted with 0-2 R and there are 0-3 ring double bonds;

E is selected from phenyl, pyridyl, pyrimidyl, pyrazinyl, and pyridazinyl, and is substituted with 1-2 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-2 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-1 carbonyl and 1-2 R and there are 0-3 ring double bonds;

R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NHC(═NR⁸)NR⁷R⁹, ONHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;

alternatively, when 2 R groups are attached to adjacent atoms, they combine to form methylenedioxy or ethylenedioxy;

R⁵, at each occurrence, is selected from CF₃, OH, C₁₋₄ alkoxy, C₁₋₆ alkyl, —(CH₂)ᵣ—C₃₋₁₀ carbocycle substituted with 0-2 R^{5a}, and —(CH₂)ᵣ-5-10 membered heterocycle containing from 1-4 heteroatoms selected from the group consisting of N, O, and S(O)ₚ, and substituted with 0-2 R^{5a};

R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣ—C(═NR⁶)NR⁶R^{6a}, (CH₂)ᵣNR⁶C(═NR⁶)NR⁶R^{6a}, (CH₂)ᵣSO₂NR⁶R^{6a}, (CH₂)ᵣNR⁶SO₂NR⁶R^{6a}, (CH₂)ᵣNR⁶SO₂—C₁₋₄ alkyl, (CH₂)ᵣNR⁶SO₂CF₃, (CH₂)ᵣNR⁶SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;

R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

alternatively, NR⁶R^{6a} forms a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;

R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;

R⁸, at each occurrence, is selected from H, C₁₋₆ alkyl, and (CH₂)ₙ-phenyl;

alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;

R⁹, at each occurrence, is selected from H, C₁₋₆ alkyl, and (CH₂)ₙ-phenyl;

alternatively, R⁴—X² is selected from:

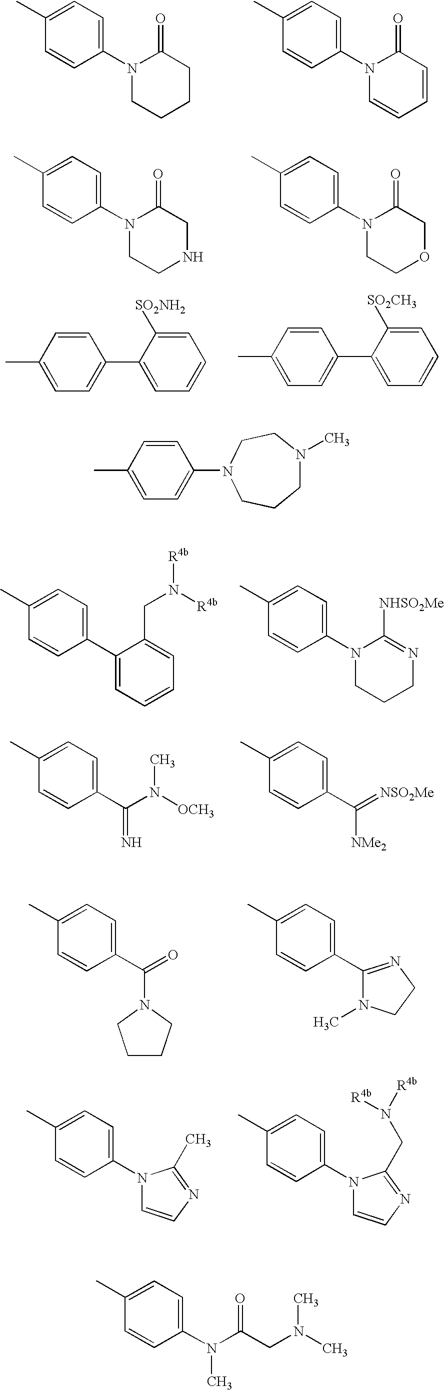

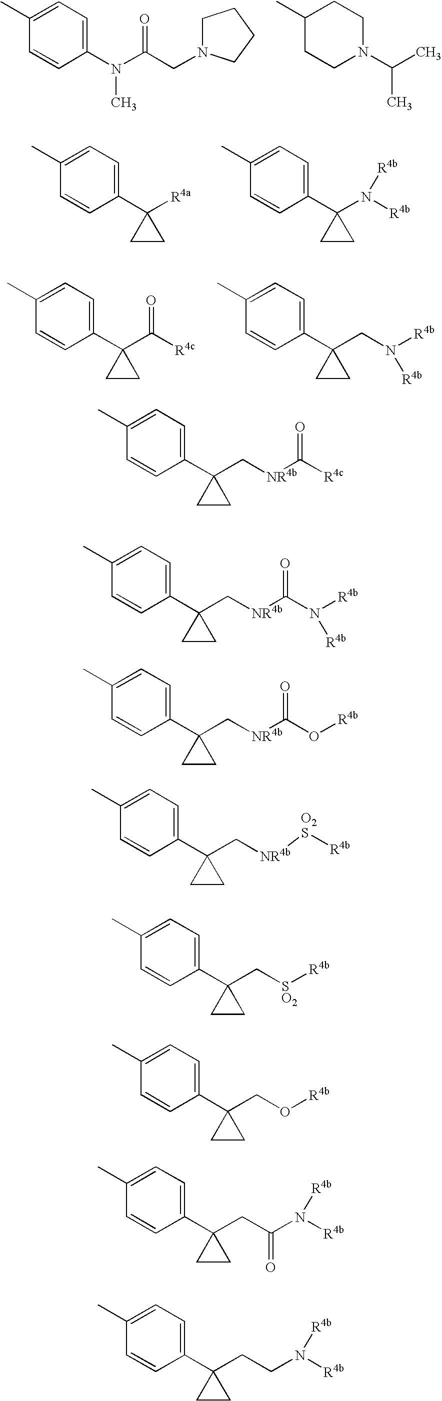

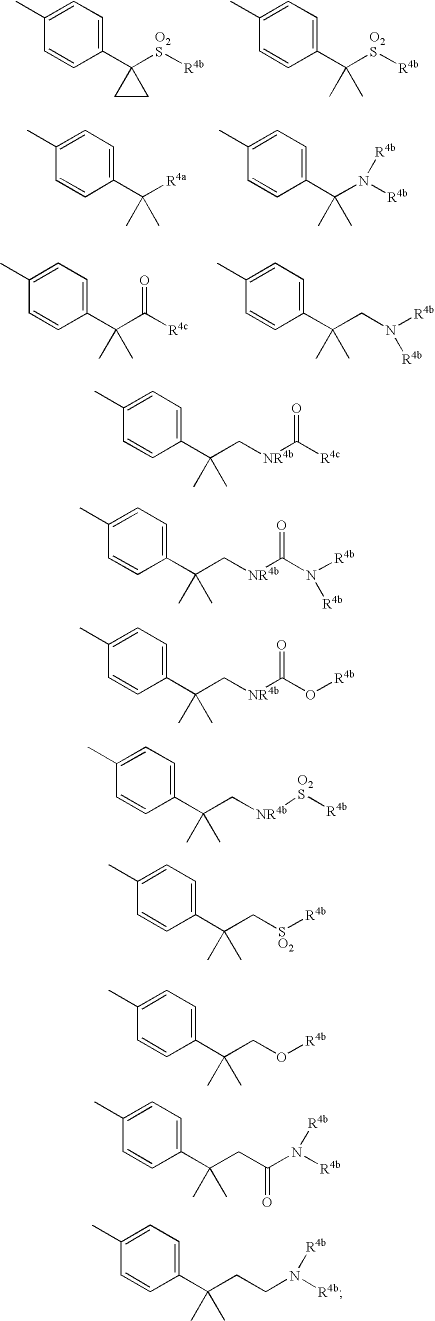

R^{4a} is substituted with 0-2 R^{4d} and selected from morpholine, 1,1-dioxo-thiomorpholine, dihydropyridine, piperidine, piperazine, pyrrolidine, imidazole, imidazoline, imidazolidine, oxazoline, and thiazoline;

R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;

R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;

R^{4d} is selected from ═O, OH, OCH₃, and CH₃;

n, at each occurrence, is selected from 0, 1, 2, and 3;

p, at each occurrence, is selected from 0, 1, and 2;

r, at each occurrence, is selected from 0, 1, 2, 3, 4, 5, and 6; and

t, at each occurrence, is selected from 0, 1, 2, and 3.

In a thirteenth embodiment, the present invention provides a novel process for preparing a compound of formula IIId, comprising:

(a₁) contacting a compound of formula Id with a compound of formula IId in the presence of a first base and a first solvent to form a cycloaddition product;

(a₂) contacting the cycloaddition product from reaction (a₁) with a first acid to form a compound of formula IIId;

wherein:

the first base is selected from a tertiary amine base and a pyridine base;

the first acid is selected from HCl, AcOH, H₂SO₄, and H₃PO₄; and

the first solvent is an aprotic solvent.

In a fourteenth embodiment, the present invention provides a novel process for preparing a compound of formula IIIe:

comprising:

(a₁) contacting a compound of formula Ib with a compound of formula IIb in the presence of a first base and a first solvent to form a cycloaddition product;

(a₂) contacting the cycloaddition product from reaction (a₁) with a first acid to form a compound of formula IIIb; 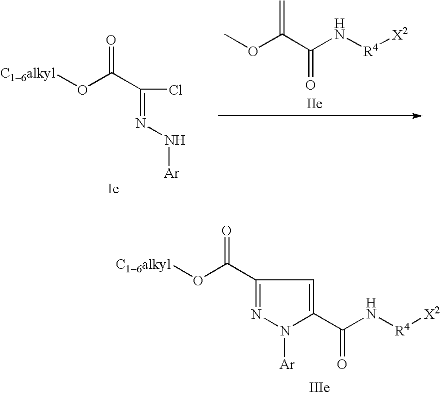
wherein:

the first base is triethylamine;

the first solvent is ethyl acetate;

the first acid is HCl;

the second base is NaOEt;

the second solvent is EtOH;

X² is I;

R⁴ is selected from phenyl, pyridyl, and pyrimidyl;

Ar is selected from phenyl, 2-fluorophenyl, 3-aminomethyl-phenyl, 3-amidino-phenyl, 3-amido-phenyl, 3-chlorophenyl, 4-methoxyphenyl, 2-naphthyl, 1-fluoro-2-naphthyl, 1-cyano-2-naphthyl, and 6-chloro-2-naphthyl;

p, at each occurrence, is selected from 0, 1, and 2;

alternatively, R⁴—X² is selected from:

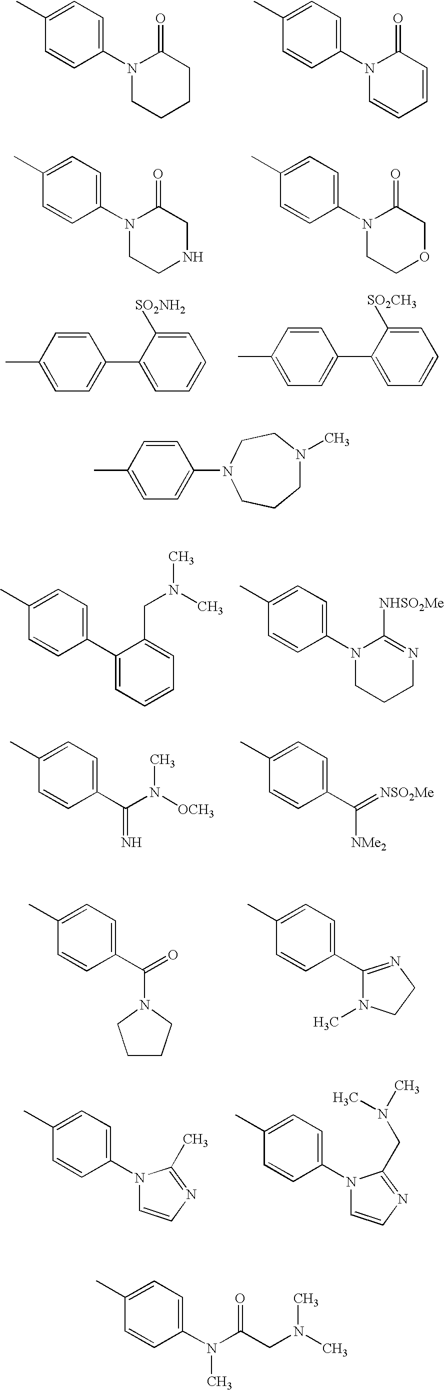

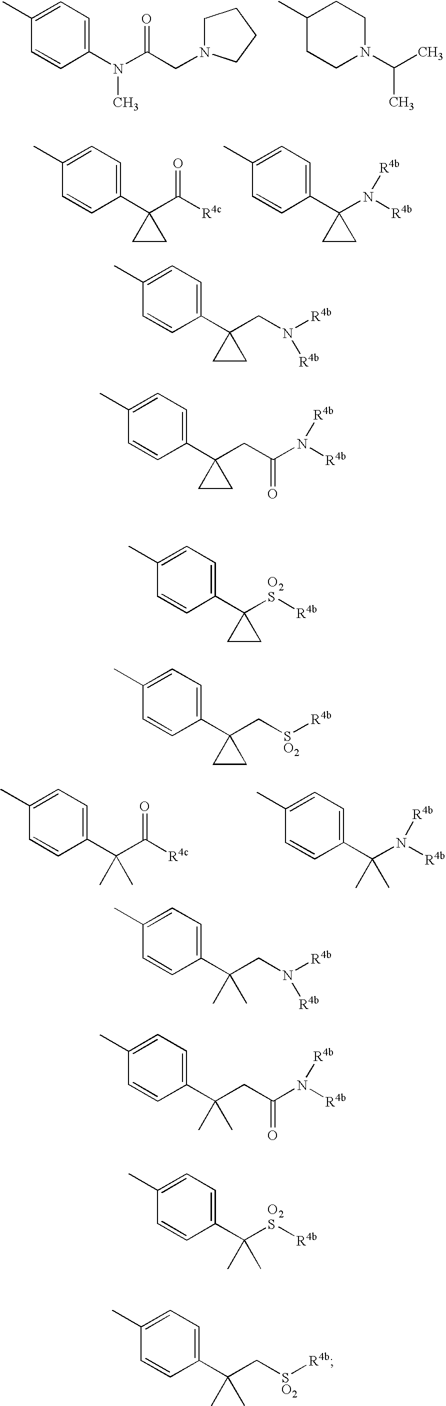

R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;

R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl; and

R^{4d} is selected from ═O, OH, OCH₃, and CH₃.

The present invention may be embodied in other specific forms without departing from the spirit or essential attributes thereof. Thus, the above embodiments should not be considered limiting. Any and all embodiments of the present invention may be taken in conjunction with any other embodiment or embodiments to describe additional embodiments. Each individual element of the embodiments is its own independent embodiment. Furthermore, any element of an embodiment is meant to be combined with any and all other elements from any embodiment to describe an additional embodiment. In addition, the present invention encompasses combinations of different embodiment, parts of embodiments, definitions, descriptions, and examples of the invention noted herein.

Definitions

All examples provided in the definitions as well as in other portions of this application are not intended to be limiting, unless stated.

The present invention can be practiced on multigram scale, kilogram scale, multikilogram scale, or industrial scale. Multigram scale, as used herein, can be in the scale wherein at least one starting material is present in 10 grams or more, at least 50 grams or more, or at least 100 grams or more. Multikilogram scale means the scale wherein more than one kilo of at least one starting material is used. Industrial scale means a scale which is other than a laboratory sale and which is sufficient to supply product sufficient for either clinical tests or distribution to consumers.

Equivalents mean molar equivalents unless otherwise specified.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, and racemic forms and all geometric isomeric forms of a structure are intended, unless the specific stereochemistry or isomeric form is specifically indicated. All processes used to prepare compounds of the present invention and intermediates made therein are considered to be part of the present invention. Tautomers of compounds shown or described herein are considered to be part of the present invention.

Examples of the molecular weight of compounds of the present invention include (a) less than about 500, 550, 600, 650, 700, 750, or 800 grams per mole, (b) 800 grams per mole, (c) less than about 750 grams per mole, and (d) less than about 700 grams per mole.

“Substituted” means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties.

The present invention includes all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present invention is also includes all stable oxides of thiol and amino groups, even when not specifically written. When an amino group is listed as a substituent, the N-oxide derivative of the amino group is also included as a substituent. When a thiol group is present, the S-oxide and S,S-dioxide derivatives are also included.

When any variable (e.g., R⁶) occurs more than one time in any constituent or formula for a compound, its definition at each occurrence is independent of its definition at every other occurrence. Thus, for example, if a group is substituted with 0-2 R⁶, then the group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

Suitable aprotic solvents include ether solvents, dimethylformamide (DMF), dimethylacetamide (DMAC), benzene, toluene, 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Alcoholic solvents can be C₁₋₆ alkyl groups with 1 hydroxy group. The alkyl groups can be linear or branched. Alcoholic solvents covers primary (e.g., methanol), secondary (e.g., isopropanol alcohol), and tertiary (e.g., 2-methyl-2-propanol) alcohols. Suitable alcoholic solvents include methanol, ethanol, 1-propanol, 2-propanol, 1-butanol, 2-butanol, 2-methyl-1-propanol, 2-methyl-2-propanol, 1-pentanol, 2-pentanol, 3-pentanol, 2,2-dimethyl-1-propanol, 3-methylbutanol, 2-methyl-2-butanol, 1-hexanol, and 2-ethyl-1-butanol.

Suitable ether solvents include dimethoxymethane, tetrahydrofuran, 1,3-dioxane, 1,4-dioxane, furan, diethyl ether, 1,2-dimethoxyethane, diethoxymethane, dimethoxymethane, ethylene glycol dimethyl ether, ethylene glycol diethyl ether, diethylene glycol dimethyl ether, diethylene glycol diethyl ether, triethylene glycol dimethyl ether, or t-butyl methyl ether.

“Tertiary amine” base includes trialkylamines wherein the three alkyl groups can be the same or different. Examples of alkyl include methyl, ethyl, n-propyl, i-propyl, n-butyl, s-butyl, t-butyl, n-pentyl, and s-pentyl. The alkyl groups on the substituted amine base also include cycloakyl groups (e.g., cyclopropyl, cyclobutyl, cyclopentyl, and cyclohexyl) and cycloalkyl-alkyl groups (e.g., cyclopropyl-methyl, cyclobutyl-methyl, cyclopentyl-methyl, and cyclohexyl-methyl). Examples of substituted amine bases include trimethylamine, triethylamine, tri-n-propylamine, diisopropylethylamine, and N-methyl-morpholine.

“Pyridine” base includes pyridine and substituted pyridines. Examples of substituted pyridines include picoline, lutidine, collidine, ethylpyridine, ethyl-methylpyridine, and dimethylaminopyridine.

“Alkyl” and “alkylene” includes both branched and straight-chain saturated aliphatic hydrocarbon groups having the specified number of carbon atoms. C₁₋₁₀ alkyl, includes C₁, C₂, C₃, C₄, C₅, C₆, C₇, C₈, C₉, and C₁₀ alkyl groups. Examples of alkyl include methyl, ethyl, n-propyl, i-propyl, n-butyl, s-butyl, t-butyl, n-pentyl, and s-pentyl. Examples of alkylene include methylene, ethylene, n-propylene, i-propylene, n-butylene, s-butylene, t-butylene, n-pentylene, and s-pentylene. “Haloalkyl” includes both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include trifluoromethyl, trichloromethyl, pentafluoroethyl, and pentachloroethyl. “Alkoxy” represents an alkyl group as defined above with the indicated number of carbon atoms attached through an oxygen bridge. C₁₋₁₀ alkoxy, includes C₁, C₂, C₃, C₄, C₅, C₆, C₇, C₈, C₉, and C₁₀ alkoxy groups. Examples of alkoxy include methoxy, ethoxy, n-propoxy, i-propoxy, n-butoxy, s-butoxy, t-butoxy, n-pentoxy, and s-pentoxy. “Cycloalkyl” includes saturated ring groups, such as cyclopropyl, cyclobutyl, or cyclopentyl. C₃₋₇ cycloalkyl includes C₃, C₄, C₅, C₆, and C₇ cycloalkyl groups. Alkenyl” includes hydrocarbon chains of either straight or branched configuration and one or more unsaturated carbon-carbon bonds that may occur in any stable point along the chain, such as ethenyl and propenyl. C₂₋₁₀ alkenyl includes C₂, C₃, C₄, C₅, C₆, C₇, C₈, C₉, and C₁₀ alkenyl groups. “Alkynyl” includes hydrocarbon chains of either straight or branched configuration and one or more triple carbon-carbon bonds that may occur in any stable point along the chain, such as ethynyl and propynyl. C₂₋₁₀ Alkynyl includes C₂, C₃, C₄, C₅, C₆, C₇, C₈, C₉, and C₁₀ alkynyl groups.

“Carbocycle” means any stable 3, 4, 5, 6, or 7-membered monocyclic or bicyclic or 7, 8, 9, 10, 11, 12, or 13-membered bicyclic or tricyclic, any of which may be saturated, partially unsaturated, or unsaturated (aromatic). When a carbocycle is referred to as an “aromatic” or “aromatic carbocycle,” this means that a fully unsaturated, i.e., aromatic, ring is present in the carbocycle. An aromatic carboocycle only requires one ring to be aromatic, if more than one ring is present (e.g., tetrahydronaphthalene). Examples of such carbocycles include cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

“Heterocycle” or “heterocyclic group” means a stable 3, 4, 5, 6, or 7-membered monocyclic or 7, 8, 9, 10, 11, or 12-membered bicyclic or tricyclic heterocyclic ring which is saturated, partially unsaturated, or unsaturated (aromatic), and which consists of carbon atoms and 1, 2, 3, 4, or 5 ring heteroatoms independently selected from the group consisting of N, O and S. Heterocycle includes any bicyclic group in which one heterocyclic ring is fused to a second ring, which may be carbocyclic (e.g. benzo fusion) or heterocyclic. When a heterocycle is referred to as an “aromatic heterocycle” or “heteroaryl,” this means that a fully unsaturated, i.e., aromatic, ring is present in the heterocycle. An aromatic heterocycle only requires one ring to be aromatic, if more than one ring is present. The aromatic portion of the aromatic heterocycle can be a carbocycle or heterocycle. The nitrogen and sulfur heteroatoms in the heterocycle may optionally be oxidized (i.e., N→O and S(O)p). The nitrogen atom may be unsubstituted (i.e., N or NH) or substituted (i.e., NR wherein R is a substituent) and may optionally be quaternized. The heterocyclic ring may be attached to its pendant group at any heteroatom or carbon atom that results in a stable structure. The heterocyclic rings described herein may be substituted on a carbon or on a nitrogen atom, if the resulting compound is stable. If the total number of S and O atoms in the heterocycle exceeds 1, then these heteroatoms can be non-adjacent. As an example, the total number of S and O atoms in the heterocycle can be 0 or 1. Bridged and spiro rings are also included in the definition of heterocycle. A bridged ring occurs when one or more atoms (i.e., C, O, N, or S) link two non-adjacent carbon or nitrogen atoms. Examples of bridges include one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro rings are formed when to or more atoms (i.e., C, O, N, or S) of a chain are attached to the same carbon atom of a heterocycle (or carbocycle if fused to a heterocycle). When a spiro ring is present, the substituents recited for the ring may also be present on the spiro.

Examples of heterocycles include acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrinidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” refer to derivatives of the disclosed compounds wherein the parent compound is modified by making acid or base salts thereof. Examples of pharmaceutically acceptable salts include mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluene sulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Examples of organic solvents include non-aqueous media (e.g., ether, ethyl acetate, ethanol, isopropanol, and acetonitrile). Lists of suitable salts are found in Remington's Pharmaceutical Sciences, 18th ed., Mack Publishing Company, Easton, Pa., 1990, p 1445, the disclosure of which is hereby incorporated by reference.

“Stable compound” and “stable structure” indicate a compound that is sufficiently robust to survive isolation to a useful degree of purity from a reaction mixture, and formulation into an efficacious therapeutic agent.

“Substituted” indicates that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Synthesis

By way of example and without limitation, the present invention may be further understood by the following schemes and descriptions.

### Preparation of 4,5-dihydro-pyrazolo[3,4-c]pyrid-2-ones

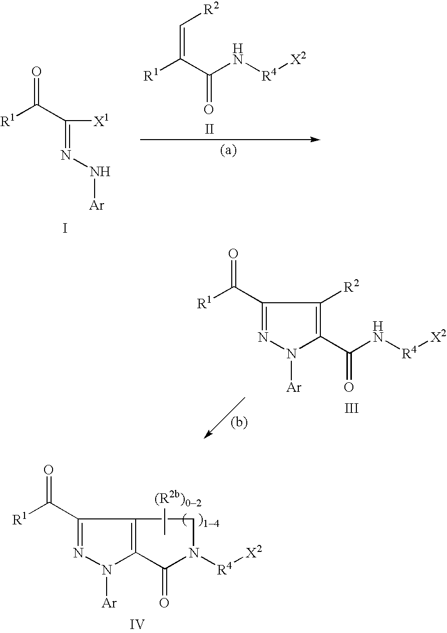 Reaction (a)

Formula III is formed by a 1,3-dipolar cycloaddition between formulas I and II, followed by an elimination to the pyrazole compound.

Cycloaddition (a₁)

The 1,3-dipolar cycloaddition between formulas I and II can be achieved by contacting formulas I and II in the presence of a base and a solvent. Examples of bases include (a) tertiary amine or a pyridine, (b) a tertiary amine, and (c) triethylamine (TEA). Examples of bases include (a) aprotic solvents, (b) toluene or ethyl acetate, and (c) ethyl acetate. Examples of reaction temperatures include (a) from room temperature up to the reflux point of the solvent used (e.g., 70° C.) and (b) from 60-100° C. The cycloaddition product can either be purified or carried directly to the next reaction without purification.

The compounds of formula II can be 2,3-dihydrofurans (e.g., R² and R³ combine to complete the dihydrofuran ring). Compounds of formula II wherein R² and R³ combine to form a dihydrofuran ring can be prepared from 2,3-dihydrofuran and an appropriately substituted R⁴-isocyanate (e.g., phenyl isocyanate or 4-iodophenyl isocyanate). This addition can generally be accomplished in an aprotic solvent (e.g., THF) and in the presence of a strong base (e.g., an alkyl lithium). The compound of formula II can be formed by cooling 2,3-dihydrofuran in an aprotic solvent (e.g., −78° C.), followed by addition of a strong base (e.g., t-butyl lithium). An appropriate isocyanate can then be added to the cooled solution.

Elimination (a₂)

Elimination to the pyrazole compound can be effected in the presence of a protic acid. Examples of protic acids include (a) HCl, AcOH, H₂SO₄, and H₃PO₄ and (b) HCl. Examples of solvents include (a) an aprotic solvent, (b) toluene and ethyl acetate, and (c) ethyl acetate. The elimination can be run in the same solvent as the cycloaddition. Examples of reaction temperatures include (a) from room temperature up to the reflux point of the solvent used (e.g., 70° C.) and (b) from room temperature to 100° C.

Reaction (b)

Formula IV is formed from formula III by cyclization. Specifically, the amide nitrogen of formula III displaces the terminal leaving group X³ of R². Thus, X³ is a leaving group capable of being displaced by the amide nitrogen of formula III. The reaction sequence for reaction (b) is dependent upon the terminal group of R².

Functional Group Conversion and Cyclization (b₁)

When the terminal group of R² (i.e., R^{2a}) is OH, conversion to leaving group, X³ can facilitate cyclization to formula IV. There are numerous known procedures for converting a terminal OH group to a leaving group. Leaving group in this instance includes, but is not limited to, F, Cl, Br, I, OSO₂Me, OSO₂CF₃, OSO₂Ph, and OSO₂Ph-p-Me. One way of conversion is by reaction with mesyl chloride in the presence of a base. Examples of bases include (a) tertiary amine and (b) triethylamine. Examples of solvents include (a) an aprotic solvent and (b) dichloromethane.

After the hydroxyl group has been converted to an appropriate leaving group, formula IV can be formed by contacting formula III with a base in the presence of a solvent. Examples of bases include (a) alkoxides, (b) C₁₋₆ alkoxide, and (c) ethoxide. Examples of counterions for the alkoxide include (a) Li, Na, K, Li, and Mg and (b) Na. The solvent used for this cyclization can be an alcohol of the alkoxide (e.g., EtOH). Other useful solvents are aprotic. Examples of aprotic solvents include dimethylformaide (DMF) and dimethylsulfoxide (DMSO). This reaction can be run from room temperature up to the reflux point of the solvent used.

Hydroxy Cyclization (b₂)

Alternatively, formula IV can be formed without going through leaving group X³. One way of cyclizing via the hydroxyl group is by using Mitsunobo conditions. Formula IV can be formed by contacting formula III with a phosphine and a diazo reagent. Examples of phosphines include (a) tri-tert-butyl phosphine, trimethyl phosphine, trially phosphine, tritolyl phophine, triphenyl phospine, and tri-n-butyl phosphine and (b) triphenyl phosphine. Examples of diazos reagents include (a) diethyl azodicarboxylate, dibenzyl azodicarboxylate, di-tert-butyl azodicarboxylate, diisopropyl azodicarboxylate, diphenyl azodicarboxylate, and dimethyl azodicarboxylate and (b) diethyl azodicarboxylate (DEAD). This reaction can be run under inert conditions. An aprotic solvent can be used (e.g., ether or THF).
Pyridone Addition 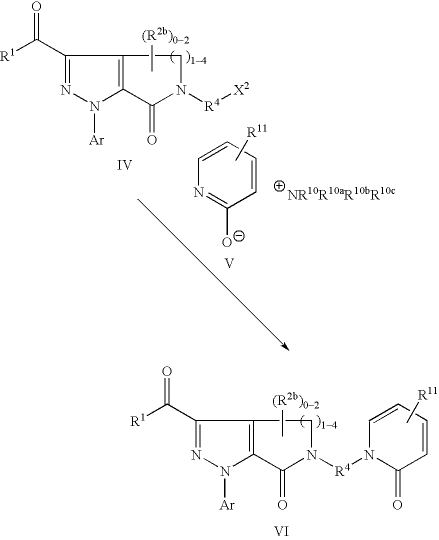
Reaction (c)

Formula VI is formed by reacting formula IV with 2-pyridinium oxide salt V. This reaction can be conducted in the presence of a metal salt catalyst. Examples of metal salt catalysts include (a) a copper salt (e.g., CuI, CuCl, CuBr, and CuOTf) or a palladium salt (e.g., PdCl₂ and Pd(OAc)₂), (b) a copper (I) salt, and (c) CuI or CuOTf. This reaction can be run in a number of solvents, including alcohols and aprotic solvents. Examples of solvents for the reaction include (a) alcohols and aprotic solvents, (b) aprotic solvents, and (c) DMF. Examples of reaction temperatures include (a) from room temperature up to the reflux point of the solvent used, (b) from about room temperature, 30, 40, 50, 60, 70, 80, 90, 100, 110, 120, 130, 140, 150, to 160° C., and (c) from room temperature to about 160° C. It may be useful to run this reaction under an inert atmosphere (e.g., nitrogen or argon).

The 2-pyridinium oxide salt, V, can be made from its corresponding hydroxy-pyridine and hydroxyl-ammonium salt (i.e., HO⁻N⁺(R¹⁰R^{10a}R^{10b}R^{10c}). The hydroxy-pyridine and hydroxy-ammonium salt can be contacted in toluene, benzene, or a hydrocarbon solvent (e.g., hexane or heptane), under water removing conditions. This reaction can be run from room temperature up to the reflux point of the solvent used. The 2-pyridinium oxide salt, once formed, can be used in situ or can be isolated prior to contacting with formula IV.

The 2-pyridinium oxide salt, V, can be made from its corresponding hydroxy-pyridine and ammonium salt (i.e., HO⁻N⁺(R¹⁰R^{10a}R^{10b}R^{10c}). The ammonium salt can be a hydroxide. It can be beneficial to contact the hydroxy-pyridine and hydroxy-ammonium salt in a solvent capable for forming an azeotrope (e.g., toluene and benzene) under water removing conditions (e.g., Dean-Stark apparatus or distallation). This reaction can be run from room temperature up to the reflux point of the solvent used. The 2-pyridinium oxide salt, once formed, can be used in situ or can be isolated prior to contacting with formula IV.

Suitable examples of ammonium hydroxides and the corresponding pyridin-2-olate include benzyltrimethylammonium hydroxide (to form benzyltrimethylammonium pyridin-2-olate), diethyldimethylammonium hydroxide (to form diethyldimethylammonium pyridin-2-olate), dimethyldodecylethylammonium hydroxide (to form dimethyldodecylethylammonium pyridin-2-olate), hexadecyltrimethylammonium hydroxide (to form hexadecyltrimethylammonium pyridin-2-olate), methyltripropylammonium hydroxide (to form methyltripropylammonium pyridin-2-olate), tetrabutylammonium hydroxide (to form tetrabutylammonium pyridin-2-olate), tetraethylammonium hydroxide (to form tetraethylammonium pyridin-2-olate), tetrahexylammonium hydroxide (to form tetrahexylammonium pyridin-2-olate), tetrakis (decyl)ammonium hydroxide (to form tetrakis (decyl)ammonium pyridin-2-olate), tetramethylammonium hydroxide (to form tetramethylammonium pyridin-2-olate), tetraoctadecylammonium hydroxide (to form tetraoctadecylammonium pyridin-2-olate), tetraoctylammonium hydroxide (to form tetraoctylammonium pyridin-2-olate), tetrapentylammonium hydroxide (to form tetrapentylammonium pyridin-2-olate), tetrapropylammonium hydroxide (to form tetrapropylammonium pyridin-2-olate), trimethylphenylammonium hydroxide (to form trimethylphenylammonium pyridin-2-olate), tributylmethylammonium hydroxide (to form tributylmethylammonium pyridin-2-olate), triethylmethylammonium hydroxide (to form triethylmethylammonium pyridin-2-olate), trihexyltetradecylammonium hydroxide (to form trihexyltetradecylammonium pyridin-2-olate), and trimethylphenylammonium hydroxide (to form trimethylphenylammonium pyridin-2-olate).

Other features of the invention will become apparent in the course of the following descriptions of examplary embodiments which are given for illustration of the invention and are not intended to be limiting thereof.

### EXAMPLES

### Example 1

### Ethyl 2-chloro-2-(2-phenylhydrazono)acetate (3a)

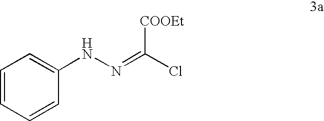

To a solution of aniline (80 mmol) in 1N HCl (70 mL) and 12N HCl (5 mL) at −5° C. was slowly added a solution of sodium nitrite (88 mmol) in water (10 mL). The mixture was stirred for 10 min at −5° C., then sodium acetate (80 mmol) was added, followed with a solution of ethyl 2-chloroacetoacetate (80 mmol) in acetone (6 mL). The mixture was allowed to warm to room temperature gradually and stirred overnight with air blowing. The precipitate was collected by vacuum filtration and dried to provide ethyl 2-chloro-2-(2-phenylhydrazono)acetate (3a) as a solid (92% yield), which was used without further purification. ¹H NMR (500 MHz, CDCl₃) δ: 1.38 (t, 3H, J=7.2), 4.38 (q, 2H, J−7.2), 7.04 (t, 1H, J=7.7), 7.21 (d, 2H, J=7.7), 7.33 (t, 2H, J=7.7), 8.34 (s, 1H).

### Example 2

### Ethyl 2-chloro-2-(2-(3-chlorophenyl)hydrazono)acetate (3b)

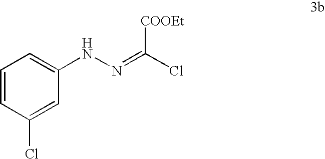

Ethyl 2-chloro-2-(2-(3-chlorophenyl)hydrazono)acetate (3b) was prepared similarly in 96% yield using 3-chloroaninline and ethyl 2-chloroacetoacetate. ¹H NMR (500 MHz, CDCl₃) δ: 1.40(t, 3H, J=7.1), 4.40(q, 2H, J=7.1), 7.01 (m, 1H), 7.06(m, 1H), 7.22 (m, 2H), 8.31 (s, 1H).

### Example 3

### 1-Chloro-1-(2-(4-methoxyphenyl)hydrazono)propan-2-one (3c)

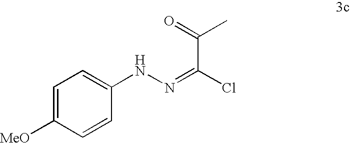

1-Chloro-1-(2-(4-methoxyphenyl)hydrazono)propan-2-one (3c) was prepared similarly in 93% yield using 4-methoxyaninline and 3-chloro-2,4-pentanedione. ¹H NMR (400 MHz, CDCl₃) δ: 2.58 (s, 3H), 3.84 (s, 3H), 7.19 (d, J=7 Hz, 2H), 7.21 (d, J=7 Hz, 2H), 8.41 (s, 1H).

### Example 4

### N-Phenyl-4,5-dihydrofuran-2-carboxamide (6a)

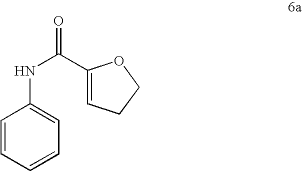

To a solution of 2,3-dihydrofuran (75 mmol) in THF (40 mL) at −78° C. was added tert-butyllithium 1.7M solution in pentane (39 mmol). The resultant solution was stirred under −60° C. for 5 min, then phenyl isocyanate (30 mmol) in THF (20 mL) was added. The reaction temperature was kept below −50° C. during the addition. The reaction was finished immediately. Ammonium chloride aqueous solution and ethyl acetate was added. The organic layer was washed with brine and dried over sodium sulfate. The solvent was removed under reduced pressure and the residue was slurried in MTBE to give product 6a as a solid (81% yield). ¹H NMR (400 MHz, CDCl₃) δ: 2.84 (m, 2H), 4.56 (t, 2H, J=9.6), 6.03 (t, 1H, J=3.1), 7.14 (t, 1H, J=7.6), 7.35 (m, 2H), 7.60 (m, 2H), 8.05 (s, 1H).

### Example 5

### N-(4-Iodophenyl)-4,5-dihydrofuran-2-carboxamide (6b)

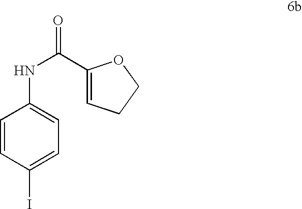

N-(4-Iodophenyl)-4,5-dihydrofuran-2-carboxamide (6b) was prepared similarly in 82% yield using 4-iodophenyl isocyanate and 2,3-dihydrofuran. ¹H NMR (500 MHz, DMSO) δ: 2.76 (dt, 2H, J=2.8, 5.8), 4.49 (t, 2H, J=9.3), 5.93 (t, 1H, J=2.8), 7.57 (dd, 2H, J=8.8, 2.9), 7.62 (dd, 2H, J=4.4, 2.8), 9.88 (s, 1H).

### Example 6

### N-(4-methoxyphenyl)-4,5-dihydrofuran-2-carboxamide (6c)

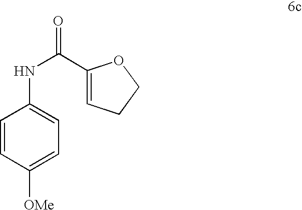

N-(4-Methoxyphenyl)-4,5-dihydrofuran-2-carboxamide (6c) was prepared similarly in 89% yield using 4-methoxyphenyl isocyanate and 2,3-dihydrofuran. ¹H NMR (500 MHz, DMSO) δ: 2.75 (dt, 2H, J=2.8, 7.1), 3.71 (d, 3H, J=6.0), 4.48 (t, 2H, J=9.4), 5.86 (t, 1H, J=2.7), 6.85 (d, 2H, J=9.4), 7.60 (d, 2H, J=8.8), 9.63 (s, 1H).

### Example 7

### Ethyl 2-(2-(phenylcarbamoyl)-4,5-dihydrofuran-3-yl)-2-(2-phenylhydrazono)acetate (7a)

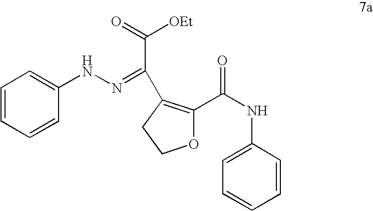

To a solution of 6a (6 mmol) and 3a (12 mmol) in EtOAc (25 mL) was added triethylamine (2.5 mL). The solution was heated at 70° C. for 15 hr before being quenched with water and EtOAc. The organic layer was washed with NaHCO₃ aqueous solution and brine, then dried over Na₂SO₄. The solvent was removed under reduced pressure to give 7a which was used directly in the next step without purification. M/z 380.18 [M+H]⁺.

### Example 8

### Ethyl 2-(2-((4-iodophenyl)carbamoyl)-4,5-dihydrofuran-3-yl)-2-(2-phenylhydrazono)acetate (7b)

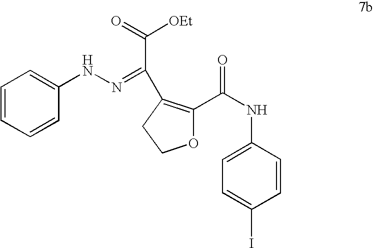

Ethyl 2-(2-((4-iodophenyl)carbamoyl)-4,5-dihydrofuran-3-yl)-2-(2-phenylhydrazono)acetate (7b) was prepared similarly and the crude product was used directly in the next step without purification. M/z 506.23 [M+H]⁺.

### Example 9

### Ethyl 2-(2-((4-methoxyphenyl)carbamoyl)-4,5-dihydrofuran-3-yl)-2-(2-phenylhydrazono)acetate (7c)

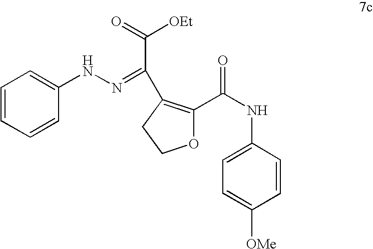

Ethyl 2-(2-((4-methoxyphenyl)carbamoyl)-4,5-dihydrofuran-3-yl)-2-(2-phenylhydrazono)acetate (7c) was prepared similarly and the crude product was used directly in the next step without purification. M/z 410.41 [M+H]⁺.

### Example 10

### Ethyl 2-(2-(3-chlorophenyl)hydrazono)-2-(2-(phenylcarbamoyl)-4,5-dihydrofuran-3-yl)acetate (7d)

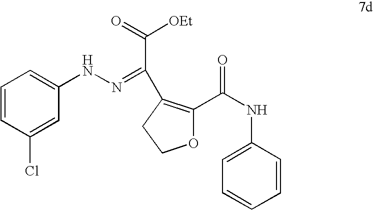

Ethyl 2-(2-(3-chlorophenyl)hydrazono)-2-(2-(phenylcarbamoyl)-4,5-dihydrofuran-3-yl)acetate (7d) was prepared similarly and the crude product was used directly in the next step without purification. M/z 414.32 [M+H]⁺.

### Example 11

### Ethyl 2-(2-((4-iodophenyl)carbamoyl)-4,5-dihydrofuran-3-yl)-2-(2-(3-chlorophenyl)hydrazono)acetate (7e)

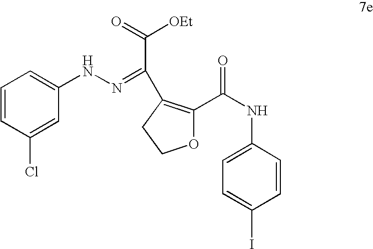

Ethyl 2-(2-((4-iodophenyl)carbamoyl)-4,5-dihydrofuran-3-yl)-2-(2-(3-chlorophenyl)hydrazono)acetate (7e) was prepared similarly, and the crude product could be recrystallized in methanol to give 7e in 70% yield. ¹H NMR (500 MHz, CDCl₃) δ: 1.38 (t, 3H, J=7.15), 2.40 (m, 2H), 3.72 (m, 1H), 4.23 (d, 1H, J=9.80), 4.35 (m, 3H), 6.94 (m, 1H), 7.13 (m, 2H), 7.31 (d, 2H, J=8.8), 7.37 (d, 1H, J−2.2), 7.62 (d, 2H, J=8.8), 8.61 (s, 1H).

### Example 12

### Ethyl 2-(2-((4-methoxyphenyl)carbamoyl)-4,5-dihydrofuran-3-yl)-2-(2-(3-chlorophenyl)hydrazono)acetate (7f)

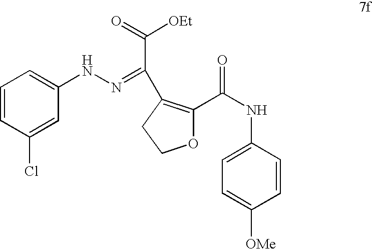

Ethyl 2-(2-((4-methoxyphenyl)carbamoyl)-4,5-dihydrofuran-3-yl)-2-(2-(3-chlorophenyl)hydrazono)acetate (7f) was prepared similarly, and the crude product could be recrystallized in methanol to give 7f in 78% yield. ¹H NMR(500 MHz, CDCl₃) δ: 1.38 (t, 3H, J=7.2), 2.41 (m, 2H), 3.73 (m, 1H), 3.78 (s, 3H), 4.26 (dd, 1H, J=2.2, 7.0), 4.35 (m, 3H), 6.86 (dd, 2H, J−3.3, 8.8), 6.95 (d, 1H, J=7.7), 7.16 (m, 2H), 7.44 (m, 3H), 8.56 (s, 1H).

### Example 13

### 1-(2-((4-Iodophenyl)carbamoyl)-4,5-dihydrofuran-3-yl)-1-(2-(4-methoxyphenyl)hydrazono)propanone (7g)

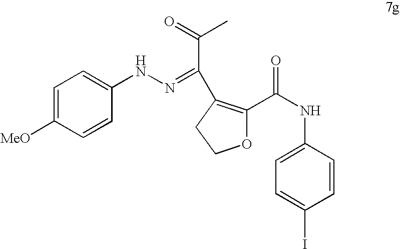

1-(2-((4-Iodophenyl)carbamoyl)-4,5-dihydrofuran-3-yl)-1-(2-(4-methoxyphenyl)hydrazono)propanone (7g) was prepared similarly, and the crude product could be recrystallized in methanol to give 7g in 88% yield. ¹H NMR (500 MHz, CDCl₃) δ: 2.37 (m, 2H), 2.49 (s, 3H), 3.75 (s, 3H), 3.80 (m, 1H), 4.19 (d, 1H, J=8.3), 4.37 (t, 1H, J=7.2), 6.82 (d, 2H, J=8.8), 7.28 (d, 2H, J=8.8), 7.32 (d, 2H, J=8.8), 7.62 (d, 2H, J=8.8), 8.62 (s, 1H).

### Example 14

### 1-(2-((4-Methoxyphenyl)carbamoyl)-4,5-dihydrofuran-3-yl)-1-(2-(4-methoxyphenyl)hydrazono)propanone (7h)

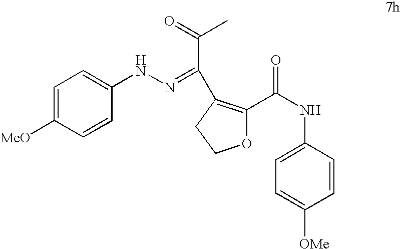

1-(2-((4-Methpxyphenyl)carbamoyl)-4,5-dihydrofuran-3-yl)-1-(2-(4-methoxyphenyl)hydrazono)propanone (7h) was prepared similarly and the crude product was used in the next step without purification. M/z 410.32 [M+H]⁺.

### Example 15

### Ethyl 4-(2-hydroxyethyl)-1-phenyl-5-(phenylcarbamoyl)-1H-pyrazole-3-carboxylate (8a)

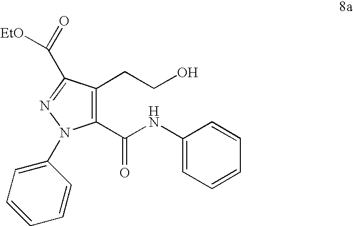

To a solution of 7a (6 mmol) in EtOAc (20 mL) and EtOH (20 mL) was added 12N HCl (6 mmol). The resulting solution was heated at 50° C. for 50 min before being quenched with water and EtOAc. The organic layer was washed with aqueous NaHCO₃ solution and brine, and then dried over Na₂SO₄. The solvent was removed under reduced pressure, and the residue was recrystallized in methanol to give solid 8a (80% yield). ¹H NMR (500 MHz, CDCl₃) δ: 1.38 (t, 3H, J−7.2), 3.14 (m, 3H), 3.99 (m, 2H), 4.38 (m, 2H), 7.11 (t, 1H, J=7.2), 7.29 (t, 2H, J=7.2), 7.38 (m, 3H), 7.48 (dd, 2H, J=1.7, 4.9), 7.61 (d, 2H, J=8.8), 10.58 (s, 1H).

### Example 16

### Ethyl 4-(2-hydroxyethyl)-5-((4-iodophenyl)carbamoyl)-1-phenyl-1H-pyrazole-3-carboxylate (8b)

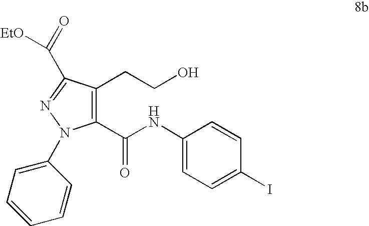

Ethyl 4-(2-hydroxyethyl)-5-((4-iodophenyl)carbamoyl)-1-phenyl-1H-pyrazole-3-carboxylate (8b) was prepared similarly in 83% yield. ¹H NMR (500 MHz, CDCl₃) δ: 1.38 (m, 3H), 3.00 (s, br, 1H), 3.15 (m, 2H), 4.03 (m, 2H), 4.40 (m, 2H), 7.41 (m, 7H), 7.58 (m, 2H), 10.62 (s, 1H).

### Example 17

### Ethyl 4-(2-hydroxyethyl)-5-((4-methoxyphenyl)carbamoyl)-1-phenyl-1H-pyrazole-3-carboxylate (8c)

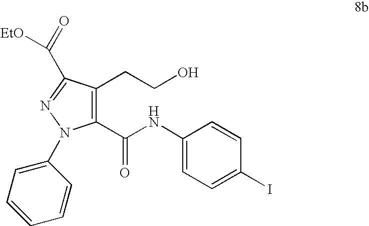

Ethyl 4-(2-hydroxyethyl)-5-((4-methoxyphenyl)carbamoyl)-1-phenyl-1H-pyrazole-3-carboxylate (8c) was prepared similarly in 86% yield. ¹H NMR (500 MHz, CDCl₃) δ: 1.37 (t, 3H, J=7.2), 3.12 (t, 2H, J=5.0), 3.35 (br, s, 1H), 3.76 (s, 3H), 3.96 (t, 2H, J−5.0), 4.38 (q, 2H, J=7.1, 7.2), 6.82 (d, 2H, J=8.8), 7.37 (m, 3H), 7.47 (m, 2H), 7.54 (d, 2H, J=9.4), 10.48 (d, 1H, J=6.1).

### Example 18

### Ethyl 1-(3-chlorophenyl)-4-(2-hydroxyethyl)-5-(phenylcarbamoyl)-1H-pyrazole-3-carboxylate (8d)

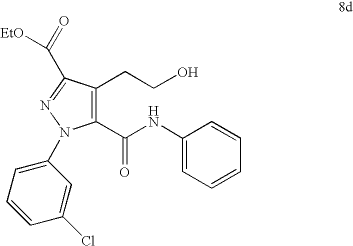

Ethyl 1-(3-chlorophenyl)-4-(2-hydroxyethyl)-5-(phenylcarbamoyl)-1H-pyrazole-3-carboxylate (8d) was prepared similarly in 60% yield, and the crude product was used directly in the next step without purification. M/z: 414.32 [M+H]⁺.

### Example 19

### Ethyl 1-(3-chlorophenyl)-4-(2-hydroxyethyl)-5-((4-iodophenyl)carbamoyl)-1H-pyrazole-3-carboxylate (8e)

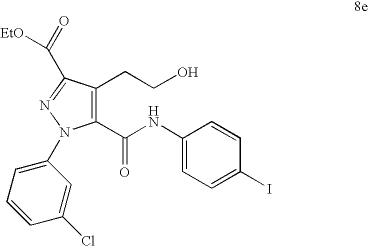

Ethyl 1-(3-chlorophenyl)-4-(2-hydroxyethyl)-5-((4-iodophenyl)carbamoyl)-1H-pyrazole-3-carboxylate (8e) was prepared similarly in 76% yield. ¹H NMR (500 MHz, CDCl₃) δ: 1.40 (m, 3H), 2.84 (s, br, 1H), 3.17 (t, 2H, J=5.5), 4.08 (t, 2H, J=5.5), 4.40 (m, 2H), 7.33 (m, 3H), 7.40 (d, 2H, J=6.8), 7.52 (s, 1H), 7.60 (m, 2H), 10.64 (m, 1H).

### Example 20

### Ethyl 1-(3-chlorophenyl)-4-(2-hydroxyethyl)-5-((4-methoxyphenyl)carbamoyl)-1H-pyrazole-3-carboxylate (8f)

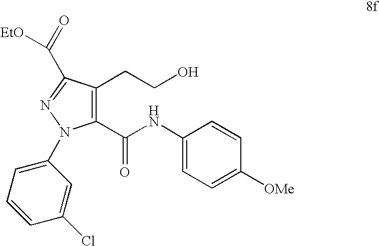

Ethyl 1-(3-chlorophenyl)-4-(2-hydroxyethyl)-5-((4-methoxyphenyl)carbamoyl)-1H-pyrazole-3-carboxylate (8f) was prepared similarly in 85% yield. ¹H NMR (500 MHz, CDCl₃) δ: 1.38 (m, 3H), 3.16 (t, 2H, J=5.0), 3.43 (d, 1H, J=3.8), 3.76 (d, 3H, J=2.7), 4.05 (t, 2H, J=4.9), 4.38 (m, 2H), 6.81 (m, 2H), 7.34 (m, 3H), 7.54 (m, 3H), 10.46 (s, 1H).

### Example 21

### 3-Acetyl-4-(2-hydroxyethyl)-N-(4-iodophenyl)-1-(4-methoxyphenyl)-1H-pyrazole-5-carboxamide (8g)

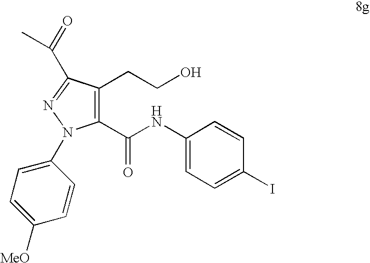

3-Acetyl-4-(2-hydroxyethyl)-N-(4-iodophenyl)-1-(4-methoxyphenyl)-1H-pyrazole-5-carboxamide (8g) was prepared similarly in 86% yield. ¹H NMR (500 MHz, CDCl₃) δ: 2.63 (s, 3H), 2.64 (s, 1H), 3.17 (t, 2H, J=5.0), 3.82 (s, 3H), 4.09 (t, 2H, J=5.0), 6.93 (d, 2H, J=8.8), 7.40 (m, 4H), 7.58 (d, 2H, J=8.8), 10.62 (s, 1H).

### Example 22

### 3-Acetyl-4-(2-hydroxyethyl)-N,1-bis(4-methoxyphenyl)-1H-pyrazole-5-carboxamide (8h)

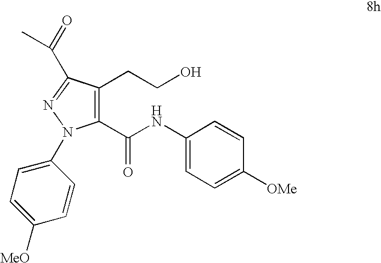

3-Acetyl-4-(2-hydroxyethyl)-N,1-bis(4-methoxyphenyl)-1H-pyrazole-5-carboxamide (8h) was prepared similarly in 71% yield. ¹H NMR (500 MHz, CDCl₃) δ: 2.63 (s, 3H), 2.81 (s, 1H), 3.17 (t, 2H, J=5.0), 3.78 (s, 3H), 3.82 (s, 3H), 4.08 (m, 2H), 6.82 (d, 2H, J=8.8), 6.93 (d, 2H, J=8.8), 7.43 (d, 2H, J=8.8), 7.55 (d, 2H, J=8.8), 10.36 (s, 1H).

### Example 23

### Ethyl 4-(2-methanesulfonyloxyethyl)-1-phenyl-5-(phenylcarbamoyl)-1H-pyrazole-3-carboxylate (9a)

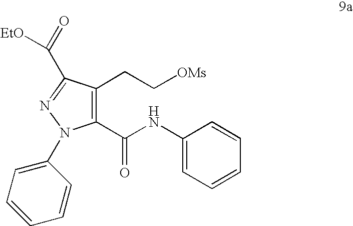

To a solution of 8a (3 mmol) in dichloromethane (25 mL) at −30° C. was added methanesulfonyl chloride (3.6 mmol) and diisopropylethylamine (3.9 mmol). The reaction was finished in seconds and quenched with aqueous NaHCO₃ and CH₂Cl₂. The organic layer was washed with 1N HCl and brine and dried over Na₂SO₄. The solvent was removed under reduced pressure, and the residue was recrystallized in methanol to give solid 9a (92% yield). ¹H NMR (500 MHz, CDCl₃) δ: 1.43 (t, 3H, J=7.1), 2.97 (s, 3H), 3.39 (t, 2H, J=5.5), 4.46 (q, 2H, J=7.1), 4.67 (t, 2H, J=5.8), 7.14 (t, 1H, J=7.7), 7.32 (t, 2H, J=7.7), 7.45 (q, 3H, J=7.6), 7.54 (q, 4H, J=8.2), 8.13 (s, 1H).

### Example 24

### Ethyl 5-((4-iodophenyl)carbamoyl)-4-(2-(methylsulfonyloxy)ethyl)-1-phenyl-1H-pyrazole-3-carboxylate (9b)

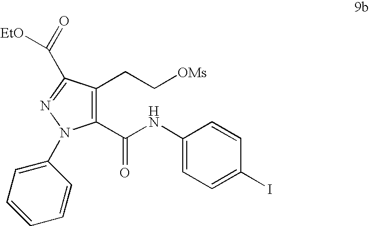

Ethyl 5-((4-iodophenyl)carbamoyl)-4-(2-(methylsulfonyloxy)ethyl)-1-phenyl-1H-pyrazole-3-carboxylate (9b) was prepared similarly in 85% yield. ¹H NMR (500 MHz, DMSO) δ: 1.32 (t, 3H, J=7.1), 3.06 (s, 3H), 3.23 (t, 2H, J=7.6), 4.36 (m, 4H), 7.66-7.37 (m, 9H), 10.84 (s, 1H).

### Example 25

### Ethyl 5-((4-methoxyphenyl)carbamoyl)-4-(2-(methylsulfonyloxy)ethyl)-1-phenyl-1H-pyrazole-3-carboxylate (9c)

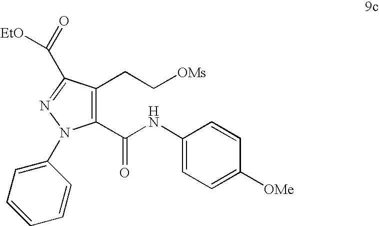

Ethyl 5-((4-methoxyphenyl)carbamoyl)-4-(2-(methylsulfonyloxy)ethyl)-1-phenyl-1H-pyrazole-3-carboxylate (9c) was prepared similarly in 100% yield. ¹H NMR (500 MHz, CDCl₃) δ: 1.41 (t, 3H, J=7.1), 2.95 (s, 3H), 3.37 (t, 2H, J=6.1), 3.76 (s, 3H), 4.43 (q, 2H, J=7.1, 7.2), 4.64 (t, 2H, J=5.5), 6.82 (d, 2H, J=8.8), 7.43 (m, 5H), 7.54 (d, 2H, J=7.2), 8.00 (s, 1H).

### Example 26

### Ethyl 1-(3-chlorophenyl)-4-(2-(methylsulfonyloxy)ethyl)-5-(phenylcarbamoyl)-1H-pyrazole-3-carboxylate (9d)

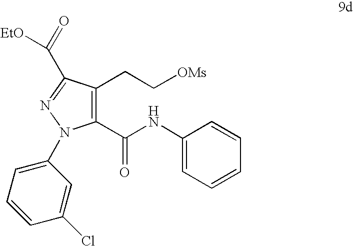

Ethyl 1-(3-chlorophenyl)-4-(2-(methylsulfonyloxy)ethyl)-5-(phenylcarbamoyl)-1H-pyrazole-3-carboxylate (9d) was prepared similarly in 85% yield. ¹H NMR (500 MHz, CDCl₃) δ: 1.42 (t, 3H, J=7.1), 2.97 (d, 3H, J=1.6), 3.34 (m, 2H), 4.43 (dq, 2H, J=1.7, 7.1), 4.66 (q, 2H, J=5.5, 3.3), 7.15 (t, 1H, J=7.2), 7.34 (m, 5H), 7.58 (d, 2H, J=7.7), 7.63 (d, 1H, J=1.7), 8.33 (s, 1H).

### Example 27

### Ethyl 1-(3-chlorophenyl)-5-((4-iodophenyl)carbamoyl)-4-(2-(methylsulfonyloxy)ethyl)-1H-pyrazole-3-carboxylate (9e)

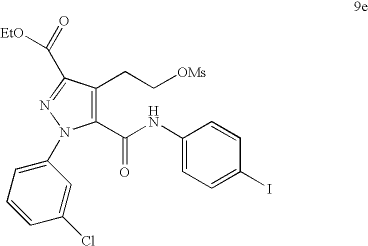

Ethyl 1-(3-chlorophenyl)-5-((4-iodophenyl)carbamoyl)-4-(2-(methylsulfonyloxy)ethyl)-1H-pyrazole-3-carboxylate (9e) was prepared similarly in 92% yield. ¹H NMR (500 MHz, DMSO) δ: 1.32 (t, 3H, J=7.2), 3.05 (s, 3H), 3.23 (t, 2H, J=6.6), 4.36 (m, 4H), 7.35 (d, 2H, J=8.8), 7.46 (m, 1H), 7.53 (d, 2H, J=5.5), 7.61 (s, 1H), 7.67 (d, 2H, J=8.8), 10.84 (s, 1H).

### Example 28

### Ethyl 1-(3-chlorophenyl)-5-((4-methoxyphenyl)carbamoyl)-4-(2-(methylsulfonyloxy)ethyl)-1H-pyrazole-3-carboxylate (9f)

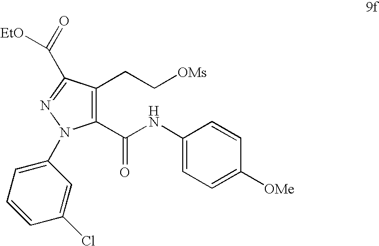

Ethyl 1-(3-chlorophenyl)-5-((4-methoxyphenyl)carbamoyl)-4-(2-(methylsulfonyloxy)ethyl)-1H-pyrazole-3-carboxylate (9f) was prepared similarly in 92% yield. ¹H NMR (500 MHz, CDCl₃) δ: 1.42 (dt, 3H, J=6.0, 1.1), 2.96 (d, 3H, J=1.6), 3.34 (t, 2H, J=5.5), 3.77 (d, 3H, J=1.6), 4.44 (m, 2H), 4.65 (t, 2H, J=5.5), 6.84 (d, 2H, J=7.2), 7.36 (m, 3H), 7.47 (d, 2H, J=7.1), 7.63 (d, 1H, J=1.6), 8.20 (s, 1H).

### Example 29

### 2-(3-Acetyl-5-((4-iodophenyl)carbamoyl)-1-(4-methoxyphenyl)-1H-pyrazol-4-yl)ethyl methanesulfonate (9g)

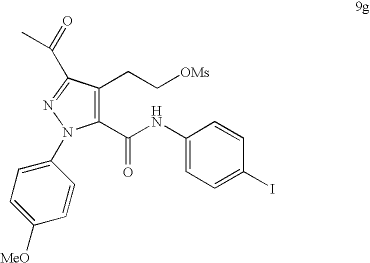

2-(3-Acetyl-5-((4-iodophenyl)carbamoyl)-1-(4-methoxyphenyl)-1H-pyrazol-4-yl)ethyl methanesulfonate (9g) was prepared similarly in 89% yield. ¹H NMR (500 MHz, CDCl₃) δ: 2.66 (s, 3H), 2.98 (s, 3H), 3.32 (t, 2H, J=5.5), 3.83 (s, 3H), 4.67 (t, 2H, J=5.5), 6.96 (d, 2H, J=8.8), 7.38 (d, 2H, J=8.8), 7.46 (d, 2H, J=8.8), 7.62 (d, 2H, J=8.8), 8.31 (s, 1H).

### Example 30

### 2-(3-Acetyl-1-(4-methoxyphenyl)-5-((4-methoxyphenyl)carbamoyl)-1H-pyrazol-4-yl)ethyl methanesulfonate (9h)

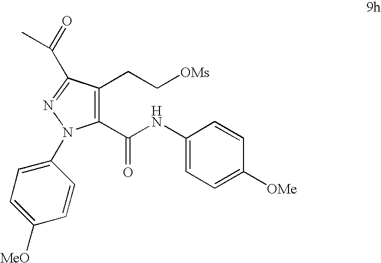

2-(3-Acetyl-1-(4-methoxyphenyl)-5-((4-methoxyphenyl)carbamoyl)-1H-pyrazol-4-yl)ethyl methanesulfonate (9h) was prepared similarly in 95% yield. ¹H NMR (500 MHz, CDCl₃) δ: 2.66 (s, 3H), 2.97 (s, 3H), 3.33 (t, 2H, J=4.5), 3.79 (s, 3H), 3.83 (s, 3H), 4.66 (t, 2H, J=5.4), 6.85 (d, 2H, J=9.2), 6.96 (d, 2H, J=8.8), 7.50 (m, 4H), 8.17 (s, 1H).

### Example 31

### Ethyl 7-oxo-1,6-diphenyl-4,5,6,7-tetrahydro-1H-pyrazolo[3,4-c]pyridine-3-carboxylate (10a)

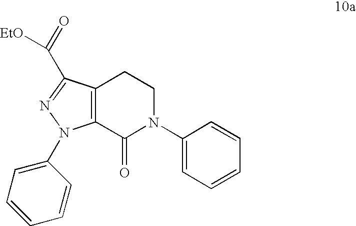

To a solution of 9a (1 mmol) in ethanol (10 mL) and DMF (3 mL) at 0° C. was added sodium ethoxide (3 mmol). The mixture was stirred at 35° C. for 30 min and then distributed between EtOAc and aqueous NH₄Cl. The organic layer was washed with water until no DMF was left. The solvent was removed under reduced pressure, and the residue was purified by slurrying in methanol to give solid 10a (87% yield). ¹H NMR (500 MHz, CDCl₃) δ: 1.43 (t, 3H, J=7.2), 3.33 (t, 2H, J=6.6), 4.12 (t, 2H, J=6.6), 4.46 (q, 2H, J=6.6), 7.21-7.42 (m, 8H), 7.56 (d, 2H, J=8.3).

### Example 32

### Ethyl 6-(4-iodophenyl)-7-oxo-1-phenyl-4,5,6,7-tetrahydro-1H-pyrazolo[3,4-c]pyridine-3-carboxylate (10b)

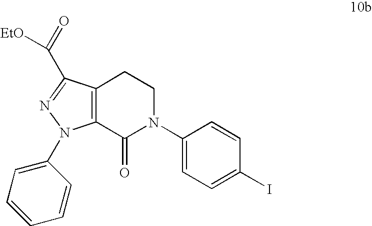

Ethyl 6-(4-iodophenyl)-7-oxo-1-phenyl-4,5,6,7-tetrahydro-1H-pyrazolo[3,4-c]pyridine-3-carboxylate (10b) was prepared similarly in 86% yield. ¹H NMR (500 MHz, CDCl₃) δ: 1.42 (t, 3H, J=7.1), 3.31(t, 2H, J=6.6), 4.09 (t, 2H, J=6.6), 4.44 (q, 2H, J=7.1), 7.07 (d, 2H, J=8.3), 7.39 (m, 3H), 7.53 (d, 2H, J=8.2), 7.67 (d, 2H, J=8.2).

### Example 33

### Ethyl 6-(4-methoxyphenyl)-7-oxo-1-phenyl-4,5,6,7-tetrahydro-1H-pyrazolo[3,4-c]pyridine-3-carboxylate (10c)

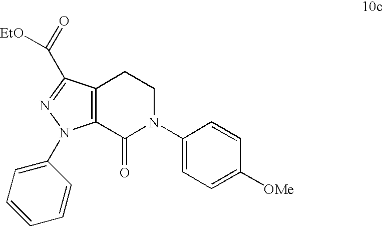

Ethyl 6-(4-methoxyphenyl)-7-oxo-1-phenyl-4,5,6,7-tetrahydro-1H-pyrazolo[3,4-c]pyridine-3-carboxylate (10c) was prepared similarly in 93% yield. ¹H NMR (500 MHz, CDCl₃) δ: 1.43 (t, 3H, J=7.1), 3.32 (t, 2H, J=6.6), 3.78 (s, 3H), 4.08 (t, 2H, J=6.6), 4.45 (q, 2H, J=7.2), 6.90 (d, 2H, J=8.8), 7.21 (m, 3H), 7.39 (m, 2H), 7.57 (t, 2H, J=1.6, 6.6).

### Example 34

### Ethyl 1-(3-chlorophenyl)-7-oxo-6-phenyl-4,5,6,7-tetrahydro-1H-pyrazolo[3,4-c]pyridine-3-carboxylate (10d)

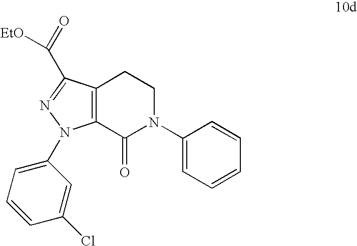

Ethyl 1-(3-chlorophenyl)-7-oxo-6-phenyl-4,5,6,7-tetrahydro-1H-pyrazolo[3,4-c]pyridine-3-carboxylate (10d) was prepared similarly in 90% yield. ¹H NMR (500 MHz, CDCl₃) δ: 1.42 (t, 3H, J=7.1), 3.32 (t, 2H, J=6.6), 4.13 (t, 2H, J=6.6), 4.46 (q, 2H, J=7.1), 7.22-7.61 (m, 9H).

### Example 35

### Ethyl 1-(3-chlorophenyl)-6-(4-iodophenyl)-7-oxo-4,5,6,7-tetrahydro-1H-pyrazolo[3,4-c]pyridine-3-carboxylate (10e)

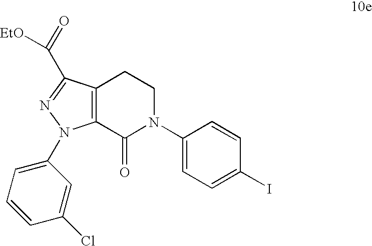

Ethyl 1-(3-chlorophenyl)-6-(4-iodophenyl)-7-oxo-4,5,6,7-tetrahydro-1H-pyrazolo[3,4-c]pyridine-3-carboxylate (10e) was prepared similarly in 90% yield. ¹H NMR (CDCl₃): δ 7.66-7.60 (m, 3H); 7.48-7.45 (m, 1H); 7.36-7.29 (m, 2H); 7.05 (d, J=8.7 Hz, 2H); 4.44(dd, J=7.08 Hz, 2H); 4.06(t, J=6.7 Hz, 2H); 3.29(t, J=6.6 Hz, 2H), 1.41(t, J=7.1 Hz, 3H).

### Example 36

### Ethyl 1-(3-chlorophenyl)-6-(4-methoxyphenyl)-7-oxo-4,5,6,7-tetrahydro-1H-pyrazolo[3,4-c]pyridine-3-carboxylate (10f)

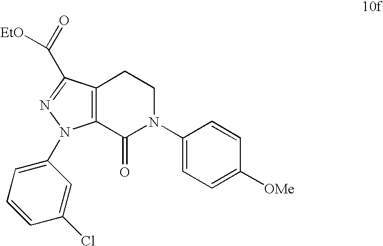

Ethyl 1-(3-chlorophenyl)-6-(4-methoxyphenyl)-7-oxo-4,5,6,7-tetrahydro-1H-pyrazolo[3,4-c]pyridine-3-carboxylate (10f) was prepared similarly in 87% yield. ¹H NMR (500 MHz, CDCl₃) δ: 1.41 (t, 3H, J=7.0), 3.29 (t, 2H, J=6.6), 3.77 (s, 3H), 4.07 (t, 2H, J=6.6), 4.44 (q, 2H, J=7.2), 6.87 (d, 2H, J=8.8), 7.19 (d, 2H, J=9.4), 7.31 (m, 2H), 7.45 (d, 1H, J=7.7), 7.59 (s, 1H).

### Example 37

### Tetrabutylammonium pyridin-2-olate

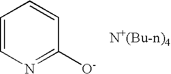

Method A: A 1 L round bottom flask was charged with 2-pyridone (47.5 g, 0.5 mol, 1 eq), tetrabutyl ammonium hydroxide (40% of aqueous solution, 324.3 g, 0.5 mol, 1 eq), and toluene (300 mL). The water was removed via a Dean-Stark apparatus. After all water was removed, the solution was cooled to rt and then to 0° C. and remained at 0° C. for 30 minutes. The slurry was filtrated under N₂ and the solid was dried under vacuum over P₂O₅ at 50° C. for 12 hours to afford the desired product as a solid (68 g, 38%).

Method B: To a 1 L round bottom flask was charged with 2-pyridone (47.5 g, 0.5 mol, 1 eq) and tetrabutyl ammonium hydroxide (40% of aqueous solution, 324.3 g, 0.5 mol, 1 eq) and toluene (300 mL). The solvent was distilled under reduced pressure at 55° C. The residual water was removed azeotropically with toluene (3×300 mL) to afford an amber oil which changed into white solid once cooled to rt. The solid was then dried under vacuum over P₂O₅ at 50° C. for 12 hours to afford the desired product as a solid (173 g, 100%).

¹H NMR (CDCl₃): δ 7.47 (m, 3H); 7.37-7.26 (m, 6H); 7.20 (dd, J=7.3, 1.7 Hz, 1H); 6.94 (ddd, J=9.2, 3.2, 2.2 Hz, 2H); 6.88 (br s, 1H), 5.73 (br s, 1H), 4.18 (t, J=6.6 Hz, 2H); 3.82 (s, 3H), 3.69 (s, 3H), 3.41 (t, J=6.6 Hz, 2H); 2.34 (s, 3H); 1.45 (br s, 1H); ¹H NMR (d₆-DMSO): δ 7.75 (s, 1H), 7.54-7.28 (m, 10H), 7.21 (d, J=7.0 Hz, 2H); 6.99 (d, J=7.3 Hz, 2H); 4.11 (br t, J=5.8 Hz, 2H); 3.81 (s, 3H); 3.55 (s, 2H); 3.34 (br s, 1H), 3.23 (br t, J=5.8 Hz, 2H); 2.22 (s, 3H); ¹³C NMR (CDCl₃): δ 167.53, 139.98, 118.68, 106.22, 58.99, 29.57, 20.01, 14.01.

### Example 38

### 1-(3-Chloro-phenyl)-7-oxo-6-[4-(2-oxo-2H-pyridin-1-yl)-phenyl]4,5,6,7-tetrahydro-3H-pyrazolo[3,4-c]pyridine-3-carboxylic acid ethyl ester (11a)

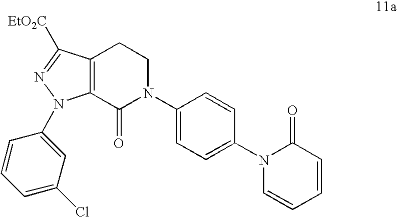

Method A: A 500 mL round bottom flask was charged with ethyl 1-(3-chlorophenyl)-6-(4-iodophenyl)-7-oxo-4,5,6,7-tetrahydro-1H-pyrazolo[3,4-c]pyridine-3-carboxylate (10e) (83.36 g, 160 mmol) and tetrabutylammonium pyridin-2-olate (107.52 g, 320 mmol). A trace of water was removed azeotropically with toluene (2×200 mL). CuI (9.12 g, 48 mmol) and 400 mL DMF were added. The reaction mixture was heated to 120° C. for 12 hours under N₂. The mixture was then cooled to rt. A solid precipitated during the cooling process. The slurry was transferred slowly to aq. NH₄OH (700 mL, 3N). The solid was collected by filtration and washed with toluene (2×350 mL). The solid was re-dissolved in CHCl₃ (500 mL) and washed with NH₄OH (3×500 mL, 3N) and H₂O (3×600 mL). The organic solution was stirred with charcoal (100 g) for 30 minutes and filtrated. The filtrate was concentrated in vacuo and triturated with EtOH to provide the desired compound (71.2 g, 90.0%) as a white solid. ¹H NMR (CDCl₃): δ 7.64-7.28 (m, 10H); 6.67(d, J=9.3 Hz, 2H); 6.27 (d, J=6.8 Hz, 2H); 6.94 (q, J=7.1 Hz, 2H); 4.20 (t, J=6.6 Hz, 2H); 3.37 (t, J=6.6 Hz, 2H), 1.46 (t, J=7.1 Hz, 3H). ¹³C NMR (CDCl₃): δ 162.33, 161.74, 156.93, 139.96, 137.64, 129.35, 129.17, 127.24, 126.27, 125.99, 124.09, 106.13, 61.42, 50.91, 21.51, 14.37.

Method B: A 50 mL round bottom flask was charged with ethyl 1-(3-chlorophenyl)-6-(4-iodophenyl)-7-oxo-4,5,6,7-tetrahydro-1H-pyrazolo[3,4-c]pyridine-3-carboxylate (10e) (521 mg, 1 mmol), 2-pyridone (190 mg, 2 mmol), tetrabutyl ammonium chloride (84 mg, 0.3 mmol), NaH (48 mg, 2 mmol), CuI (95 mg, 0.5 mmol), and DMF (5 mL) at rt under N₂, The reaction mixture was heated to 120° C. for 15 hours under N₂. The mixture was then cooled to rt. The solid was precipitated during the cooling process. The slurry was transferred slowly to aq. NH₄OH (10 mL 3N). The solid was collected by filtration and washed with toluene (2×5 mL), then H₂O (3×10 mL). The solid was dried at 60° C. in vacuo for 6 hours to provide the desired compound (380 mg, 78%) as a white solid.

¹H NMR (CDCl₃): δ 7.64-7.28 (m, 10H); 6.67(d, J=9.3 Hz, 2H); 6.27 (d, J=6.8 Hz, 2H); 6.94 (q, J=7.1 Hz, 2H); 4.20 (t, J=6.6 Hz, 2H); 3.37 (t, J=6.6 Hz, 2H), 1.46 (t, J=7.1 Hz, 3H). ¹³C NMR (CDCl₃): δ 162.33, 161.74, 156.93, 139.96, 137.64, 129.35, 129.17, 127.24, 126.27, 125.99, 124.09, 106.13, 61.42, 50.91, 21.51, 14.37.

### Example 39

### Ethyl 7-oxo-6-(4-(2-oxopyridin-1(2H)-yl)phenyl)-1-phenyl-4,5,6,7-tetrahydro-1H-pyrazolo[3,4-c]pyridine-3-carboxylate (11b)

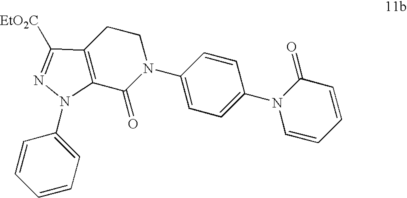

Ethyl 7-oxo-6-(4-(2-oxopyridin-1 (2H)-yl)phenyl)-1-phenyl-4,5,6,7-tetrahydro-1H-pyrazolo[3,4-c]pyridine-3-carboxylate (11b) was prepared similarly.

Numerous modifications and variations of the present invention are possible in light of the above teachings. It is therefore to be understood that within the scope of the appended claims, the invention may be practiced otherwise than as specifically described herein.

What is claimed is:

1. A process for preparing a compound of formula IV: 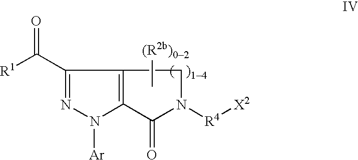
comprising:
(a) contacting a compound of formula I with a compound of formula II to form a compound of formula III; 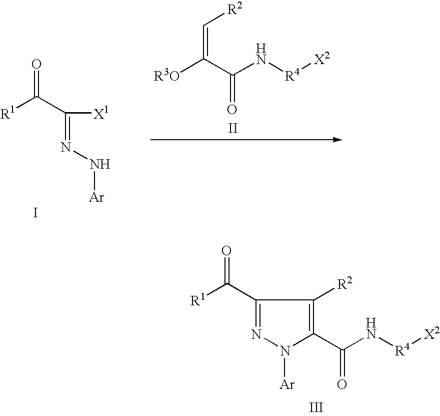
(b) cyclizing a compound of formula III to a compound of formula IV;
wherein:
X¹ is a leaving group selected from Cl, Br, and I;
X² is a leaving group selected from Cl, Br, I, OSO₂Me, OSO₂CF₃, OSO₂Ph, and OSO₂Ph-p-Me;
R¹ is selected from C₁₋₆ alkyl, C₀₋₆ alkylene-phenyl, O—C₁₋₆ alkyl, and O—C₀₋₆ alkylene-phenyl;
R² is C₁₋₄ alkylene-R^{2a}, wherein the alkylene portion of R² is substituted with 0-2 R^{2b};
R^{2a} is OH;
R^{2b} is selected from C₁₋₄ alkyl, phenyl, and benzyl;
R³ is selected from C₁₋₆ alkyl, phenyl, and benzyl;
alternatively, in formula II, R³O—*C═CH—R² forms a group selected from: 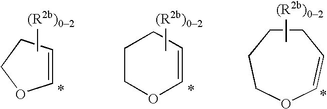
wherein * indicates the point of attachment to the remainder of formula II, provided that when R³O—*C═CH—R² forms a ring, then R² in formula III is C₂₋₄ alkylene-OH and the alkylene portion is substituted with 0-2 R^{2b};
R⁴ is a 5-10 membered aromatic carbocyclic or heterocyclic ring consisting of carbon atoms and 0-4 heteroatoms selected from O, S(O)ₚ, and N and R⁴ is substituted with 0-2 groups selected from F and C₁₋₄ alkyl;
Ar is 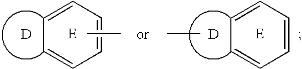
ring D, including the two atoms of Ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-2 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-2 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-1 carbonyl and 1-2 R and there are 0-3 ring double bonds;
R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NHC(═NR⁸)NR⁷R⁹, ONHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;
alternatively, when 2 R groups are attached to adjacent atoms, they combine to form methylenedioxy or ethylenedioxy;
R⁵, at each occurrence, is selected from CF₃, OH, C₁₋₄ alkoxy, C₁₋₆ alkyl, —(CH₂)ᵣ—C₃₋₁₀ carbocycle substituted with 0-2 R^{5a}, and —(CH₂)ᵣ-5-10 membered heterocycle containing from 1-4 heteroatoms selected from the group consisting of N, O, and S(O)ₚ, and substituted with 0-2 R^{5a};
R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣ—C(═NR⁶)NR⁶R^{6a}, (CH₂)ᵣNR⁶C(═NR⁶)NR⁶R^{6a}, (CH₂)ᵣSO₂NR⁶R^{6a}, (CH₂)ᵣNR⁶SO₂NR⁶R^{6a}, (CH₂)ᵣNR⁶SO₂—C₁₋₄ alkyl, (CH₂)ᵣNR⁶SO₂CF₃, (CH₂)ᵣNR⁶SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;
R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR⁶R^{6a} forms a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;
R⁸, at each occurrence, is selected from H, C₁₋₆ alkyl, and (CH₂)ₙ-phenyl;
alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
alternatively, R⁴—X² is selected from: 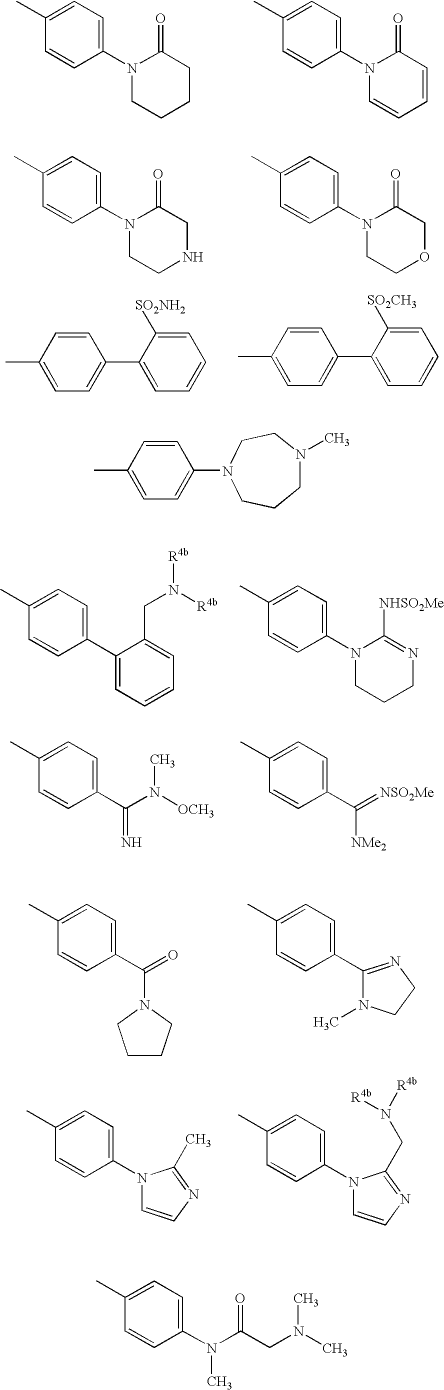 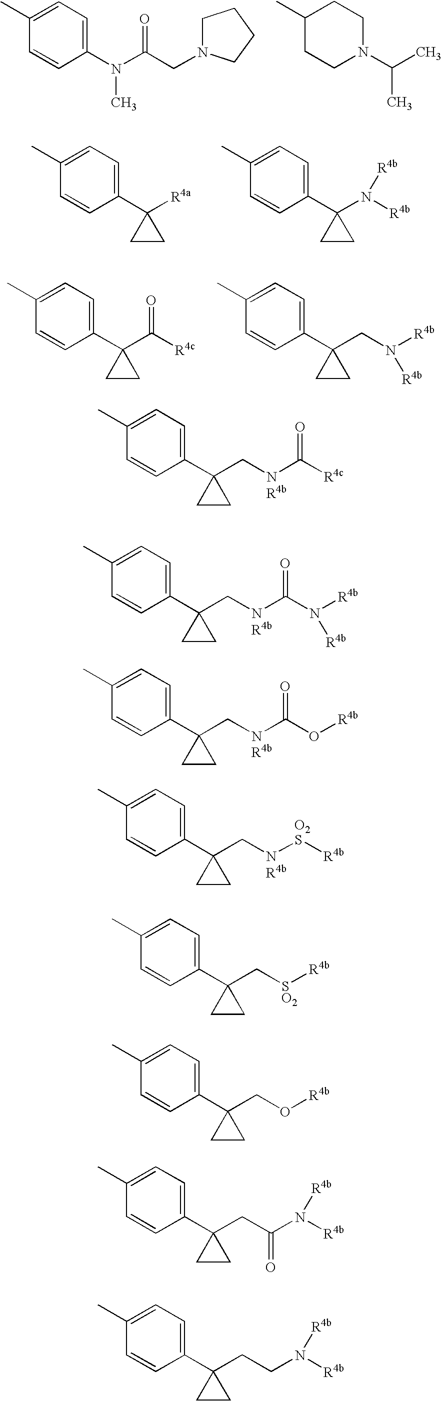 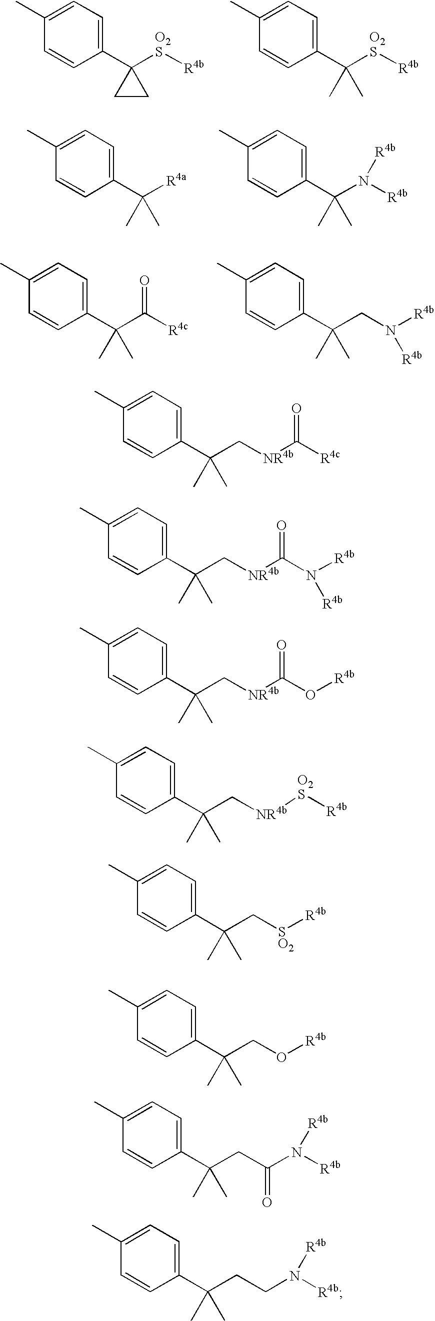
R⁴, is substituted with 0-2 R^{4d} and selected from, morpholine, 1,1-dioxo-thiomorpholine, dihydropyridine, piperidine, piperazine, pyrrolidine, imidazole, imidazoline, imidazolidine, oxazoline, and thiazoline;
R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;
R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;
R^{4d} is selected from ═O, OH, OCH₃, and CH₃;
n, at each occurrence, is selected from 0, 1, 2, and 3;
p, at each occurrence, is selected from 0, 1, and 2;
r, at each occurrence, is selected from 0, 1, 2, 3, 4, 5, and 6; and
t, at each occurrence, is selected from 0, 1, 2, and 3.

2. A process according to claim 1, the process, comprising:
(a₁) contacting a compound of formula I with a compound of formula II in the presence of a first base and a first solvent to form a cycloaddition product;
(a₂) contacting the cycloaddition product from reaction (a₁) with a first acid to form a compound of formula III;
(b) cyclizing a compound of formula III to a compound of formula IV through one of reaction sequences (b₁) or (b₂);
(b₁) converting R^{2a} of formula III to leaving group X³, followed by contacting the resulting product with a second base in the presence of a second solvent;
(b₂) alternatively, contacting a compound of formula III with a phosphine reagent and a diazo reagent under water removing conditions;
wherein:
X³ is a leaving group selected from Cl, Br, I, OSO₂Me, OSO₂CF₃, OSO₂Ph, and OSO₂Ph-p-Me;
the first base is selected from a tertiary amine base and a pyridine base;
the first acid is selected from HCl, AcOH, H₂SO₄, and H₃PO₄;
the first solvent is an aprotic solvent;
the second base is an alkoxide; and
the second solvent is selected from an alcoholic solvent and an aprotic solvent.

3. A process according to claim 1, the process, comprising: a process for preparing a compound of formula IVa: 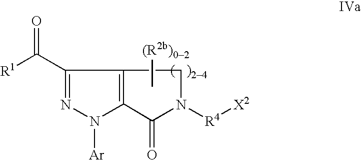
comprising:
(a₁) contacting a compound of formula Ia with a compound of formula IIa in the presence of a first base and a first solvent to form a cycloaddition product;
(a₂) contacting the cycloaddition product from reaction (a₁) with a first acid to form a compound of formula IIIa; 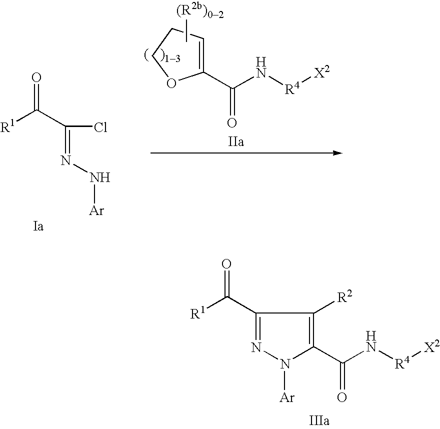
(b) cyclizing a compound of formula IIIa to a compound of formula IVa through one of reaction sequences (b₁) or (b₂);
(b₁) converting the OH group of R² in formula IIIa to leaving group X³ and contacting the resulting product with a second base in the presence of a second solvent;
(b₂) alternatively, contacting a compound of formula IIIa with a phosphine reagent and a diazo reagent under water removing conditions; wherein:
the first base is triethylamine;
the first solvent is selected from toluene and ethyl acetate;
the first acid is HCl;
the second base is a C₁₋₆ alkoxide and the counterion is selected from Li, Na, K, Li, and Mg;
the second solvent is selected from C₁₋₆ alcohol, DMF, and DMSO;
X² is a leaving group selected from Br and I;
R¹ is selected from O—C₁₋₆ alkyl and O—C₀₋₆ alkylene-phenyl;
R² is selected from C₂₋₄ alkylene-OH, wherein the alkylene portion of R² is substituted with 0-2 R^{2b};
R^{2b} is selected from C₁₋₄ alkyl, phenyl, and benzyl;
X³ is a leaving group selected from OSO₂Me, OSO₂CF₃, OSO₂Ph, and OSO₂Ph-p-Me;
R⁴ is a 5-6 membered aromatic carbocyclic or heterocyclic ring consisting of carbon atoms and 0-4 heteroatoms selected from O, S(O)ₚ, and N;
Ar is 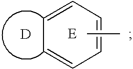
ring D, including the two atoms of Ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl and pyridyl and is substituted with 1-2 R;
alternatively, ring D is absent and ring E is selected from phenyl, pyridyl, and thienyl, and ring E is substituted with 1-2 R;
R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;
R⁵, at each occurrence, is selected from CF₃, OH, C₁₋₄ alkoxy, C₁₋₆ alkyl, —(CH₂)ᵣC₅₋₆ carbocycle substituted with 0-2 R^{5a}, and —(CH₂)ᵣ-5-6 membered heterocycle containing from 1-4 heteroatoms selected from the group consisting of N, O, and S(O)ₚ, and substituted with 0-2 R^{5a};
R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣSO₂NR⁶R^{6a}, CH₂)ᵣS(O)ₚ—C₁₋₄ alkyl, (CH₂)ᵣS(O)ₚ-phenyl, and (CH₂)ᵣ(CF₂)ᵣCF₃;
R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR⁶R^{6a} form a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;
R⁸, at each occurrence, is selected from H, C₁₋₆ alkyl, and (CH₂)ₙ-phenyl;
alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
alternatively, R⁴—X² is selected from: 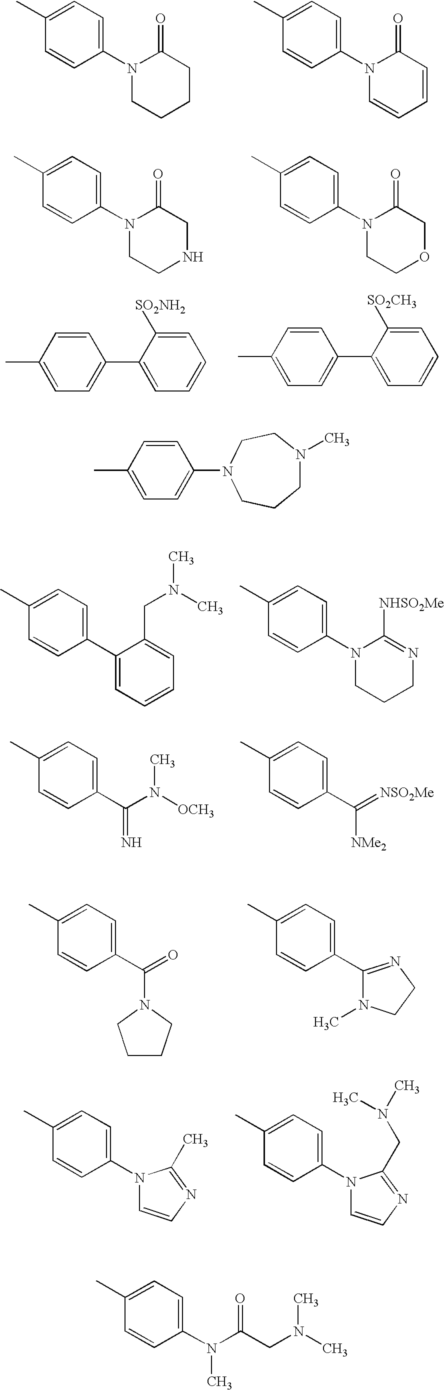 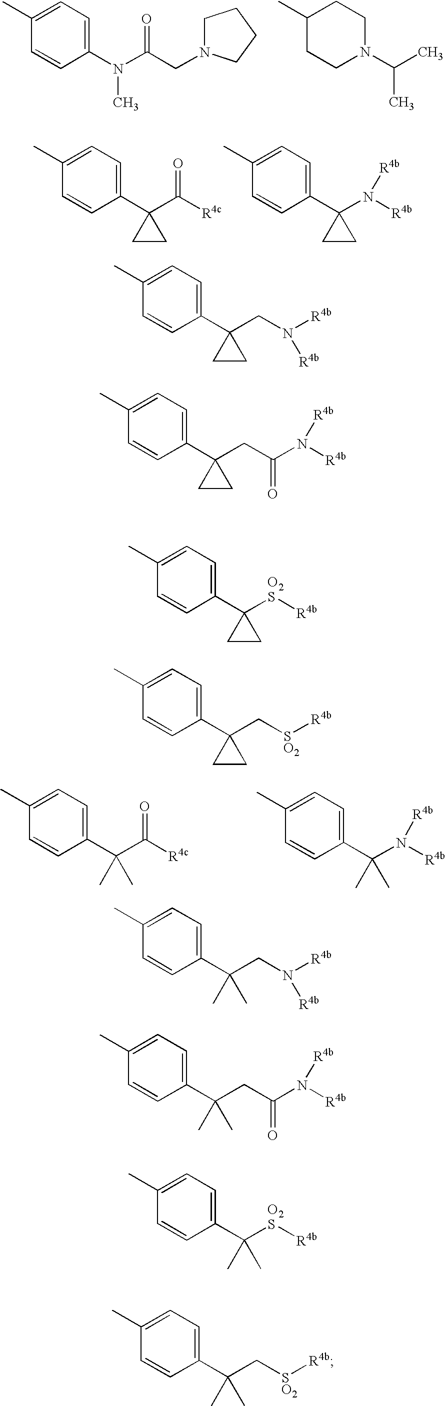
R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;
R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;
R^{4d} is selected from ═O, OH, OCH₃, and CH₃;
n, at each occurrence, is selected from 0, 1, 2, and 3;
p, at each occurrence, is selected from 0, 1, and 2;
r, at each occurrence, is selected from 0, 1, 2, and 3; and
t, at each occurrence, is selected from 0, 1, 2, and 3.

4. A process according to claim 1, the process, comprising: a process for preparing a compound of formula IVb: 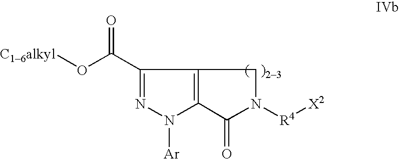
comprising:
(a₁) contacting a compound of formula Ib with a compound of formula IIb in the presence of a first base and a first solvent to form a cycloaddition product;
(a₂) contacting the cycloaddition product from reaction (a₁) with a first acid to form a compound of formula IIIb; 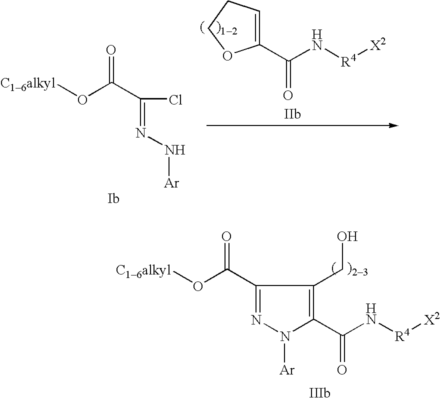
(b₂) converting a compound of formula IIIb to formula IIIb₁ and contacting the compound of formula IIIb₁ with a second base in the presence of a second solvent to form a compound of formula IVb; 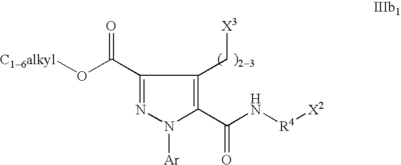
wherein:
the first base is triethylamine;
the first solvent is ethyl acetate
the first acid is HCl;
the second base is NaOEt;
the second solvent is EtOH;
X² is I;
X³ is a leaving group selected from OSO₂Me and OSO₂Ph-p-Me;
R⁴ is selected from phenyl, pyridyl, and pyrimidyl;
Ar is selected from phenyl, 2-fluorophenyl, 3-aminomethyl-phenyl, 3-amidino-phenyl, 3-amido-phenyl, 3-chlorophenyl, 4-methoxyphenyl, 2-naphthyl, 1-fluoro-2-naphthyl, 1-cyano-2-naphthyl, and 6-chloro-2-naphthyl; and
p, at each occurrence, is selected from 0, 1, and 2.

5. A process according to claim 1, the process, comprising: a process for preparing a compound of formula IVc: 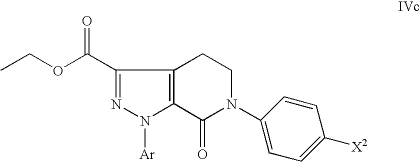
comprising:
(a₁) contacting a compound of formula Ic with a compound of formula IIc in the presence of a triethyl amine and a ethyl acetate to form a cycloaddition product;
(a₂) contacting the cycloaddition product from reaction (a₁) with a HCl to form a compound of formula IIIc; 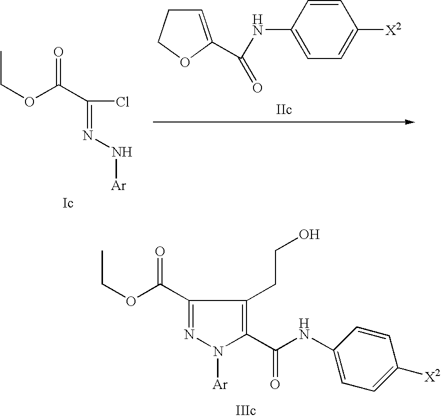
(b₁) converting a compound of formula IIIc to formula IIIc₁ and contacting the compound of formula IIIc₁ with a NaOEt in the presence of a EtOH to form a compound of formula IVc; 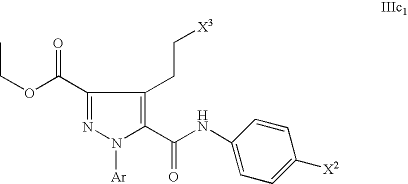
wherein:
X² is I;
X³ is OSO₂Me; and
Ar is selected from phenyl, 2-fluorophenyl, 3-chlorophenyl, and 4-methoxyphenyl.

6. A process according to claim 5, wherein the compound of formula IIIc is converted to the compound of formula IIIc_{1A} by contacting formula IIIc with mesyl chloride in the presence of a third base and a third solvent; 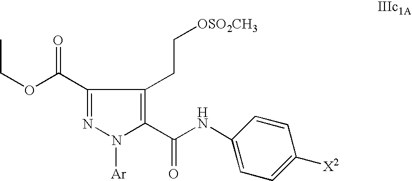
wherein:
the third base is a tertiary amine base; and
the third solvent is an aprotic solvent.

7. A process according to claim 6, wherein:
the third base is a triethylamine; and
the third solvent is dichloromethane.

8. A process for preparing a compound of formula VI: 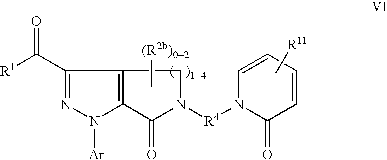
comprising:
(c) contacting a compound of formula IV with a compound of formula V in the presence of a metal salt and a fourth solvent; 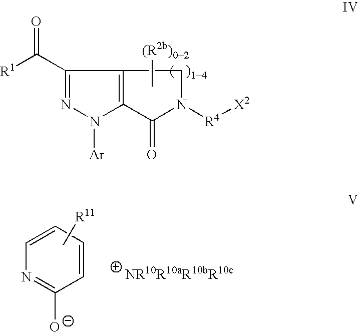
wherein:
metal salt is selected from a copper and a palladium salt;
the fourth solvent is an alcoholic or an aprotic solvent;
X² is a leaving group selected from Cl, Br, I, OSO₂Me, OSO₂CF₃, OSO₂Ph, and OSO₂Ph-p-Me;
R¹ is selected from C₁₋₆ alkyl, C₀₋₆ alkylene-phenyl, O—C₁₋₆ alkyl, and O—C₀₋₆ alkylene-phenyl;
R^{2b} is selected from C₁₋₄ alkyl, phenyl, and benzyl;
R⁴ is a 5-10 membered aromatic carbocyclic or heterocyclic ring consisting of carbon atoms and 0-4 heteroatoms selected from O, S(O)ₚ, and N and R⁴ is substituted with 0-2 groups selected from F and C₁₋₄ alkyl;
Ar is 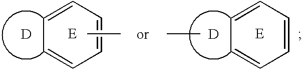
ring D, including the two atoms of Ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-2 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-2 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-1 carbonyl and 1-2 R and there are 0-3 ring double bonds;
R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NHC(═NR⁸)NR⁷R⁹, ONHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;
alternatively, when 2 R groups are attached to adjacent atoms, they combine to form methylenedioxy or ethylenedioxy;
R⁵, at each occurrence, is selected from CF₃, OH, C₁₋₄ alkoxy, C₁₋₆ alkyl, —(CH₂)ᵣ—C₃₋₁₀ carbocycle substituted with 0-2 R^{5a}, and —(CH₂)ᵣ-5-10 membered heterocycle containing from 1-4 heteroatoms selected from the group consisting of N, O, and S(O)ₚ, and substituted with 0-2 R^{5a};
R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣ—C(═NR⁶)NR⁶R^{6a}, (CH₂)ᵣNR⁶C(═NR⁶)NR⁶R^{6a}, (CH₂)ᵣSO₂NR⁶R^{6a}, (CH₂)ᵣNR⁶SO₂NR⁶R^{6a}, (CH₂)ᵣNR⁶SO₂—C₁₋₄ alkyl, (CH₂)ᵣNR⁶SO₂CF₃, (CH₂)ᵣNR⁶SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;
R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR⁶R^{6a} form a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;
R⁸, at each occurrence, is selected from H, C₁₋₆ alkyl, and (CH₂)ₙ-phenyl;
alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
R¹⁰ is selected from C₁₋₂₀ alkyl, phenyl, and benzyl;
R^{10a} is selected from C₁₋₁₈ alkyl, phenyl, and benzyl;
R^{10c} is selected from C₁₋₁₈ alkyl, phenyl, and benzyl;
R^{10c} is selected from C₁₋₁₈ alkyl, phenyl, and benzyl;
R¹¹ is selected from H, C₁₋₄ alkyl, OC₁₋₄, alkyl, F, Br, Cl, CN, NO₂, phenyl, and benzyl;
n, at each occurrence, is selected from 0, 1, 2, and 3;
p, at each occurrence, is selected from 0, 1, and 2;
r, at each occurrence, is selected from 0, 1, 2, 3, 4, 5, and 6; and
t, at each occurrence, is selected from 0, 1, 2, and 3.

9. A process according to claim 8, the process, comprising: a process for preparing a compound of formula VIa: 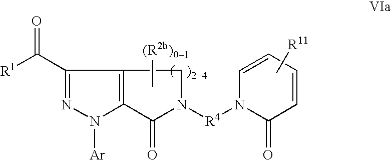
comprising:
(c) contacting a compound of formula IVa with a compound of formula V in the presence of a metal salt and a fourth solvent; 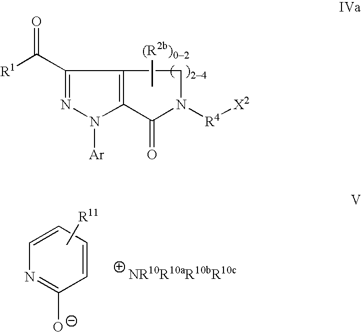
wherein:
metal salt is a copper (I) salt;
the fourth solvent is an aprotic solvent;
X² is a leaving group selected from Br and I;
R¹ is selected from O—C₁₋₆ alkyl and O—C₀₋₆ alkylene-phenyl;
R^{2b} is selected from C₁₋₄ alkyl, phenyl, and benzyl;
R⁴ is a 5-6 membered aromatic carbocyclic or heterocyclic ring consisting of carbon atoms and 0-4 heteroatoms selected from O, S(O)ₚ, and N;
Ar is 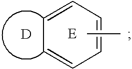
ring D, including the two atoms of Ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl and pyridyl and is substituted with 1-2 R;
alternatively, ring D is absent and ring E is selected from phenyl, pyridyl, and thienyl, and ring E is substituted with 1-2 R;
R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;
R⁵, at each occurrence, is selected from CF₃, OH, C₁₋₄ alkoxy, C₁₋₆ alkyl, —(CH₂)ᵣ-C₅₋₆ carbocycle substituted with 0-2 R^{5a}, and —(CH₂)ᵣ-5-6 membered heterocycle containing from 1-4 heteroatoms selected from the group consisting of N, O, and S(O)ₚ, and substituted with 0-2 R^{5a};
R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣSO₂NR⁶R^{6a}, CH₂)ᵣS(O)ₚ—C₁₋₄ alkyl, (CH₂)ᵣS(O)ₚ-phenyl, and (CH₂)ᵣ(CF₂)ᵣCF₃;
R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR⁶R^{6a} form a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;
R⁸, at each occurrence, is selected from H, C₁₋₆ alkyl, and (CH₂)ₙ-phenyl;
alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
R¹⁰ is selected from C₁₋₆ alkyl, phenyl, and benzyl;
R^{10a} is selected from C₁₋₆ alkyl, phenyl, and benzyl;
R^{10c} is selected from C₁₋₆ alkyl, phenyl, and benzyl;
R^{10c} is selected from C₁₋₆ alkyl, phenyl, and benzyl;
R¹¹ is selected from H, C₁₋₄ alkyl, OC₁₋₄ alkyl, F, Br, Cl, and benzyl;
n, at each occurrence, is selected from 0, 1, 2, and 3;
p, at each occurrence, is selected from 0, 1, and 2;
r, at each occurrence, is selected from 0, 1, 2, and 3; and
t, at each occurrence, is selected from 0, 1, 2, and 3.

10. A process according to claim 8, the process, comprising: a process for preparing a compound of formula VIb: 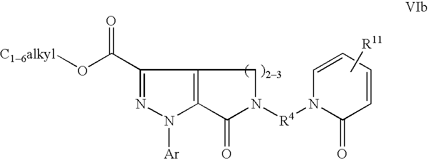
comprising:
(c) contacting a compound of formula IVb with a compound of formula V in the presence of a metal salt and a fourth solvent; 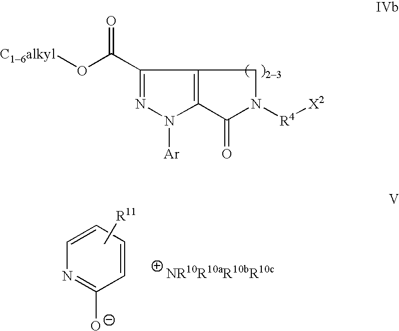
wherein:
metal salt is selected from CuI and CuOTf;
the fourth solvent is DMF;
X² is I;
R⁴ is a 6 membered aromatic carbocyclic or heterocyclic ring consisting of carbon atoms and 0-2 N atoms;
Ar is selected from phenyl, 2-fluorophenyl, 3-aminomethyl-phenyl, 3-amidino-phenyl, 3-amido-phenyl, 3-chlorophenyl, 4-methoxyphenyl, 2-naphthyl, 1-fluoro-2-naphthyl, 1-cyano-2-naphthyl, and 6-chloro-2-naphthyl;
R¹⁰ is selected from C₁₋₆ alkyl;
R^{10a} is selected from C₁₋₆ alkyl;
R^{10c} is selected from C₁₋₆ alkyl;
R^{10c} is selected from C₁₋₆ alkyl;
R¹¹ is H; and
p, at each occurrence, is selected from 0, 1, and 2.

11. A process according to claim 8, the process, comprising: a process for preparing a compound of formula VIc: 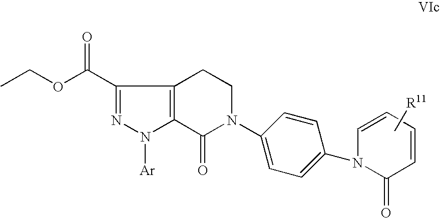
comprising:
(c) contacting a compound of formula IVc with a compound of formula V in the presence of a CuI and a DMF; 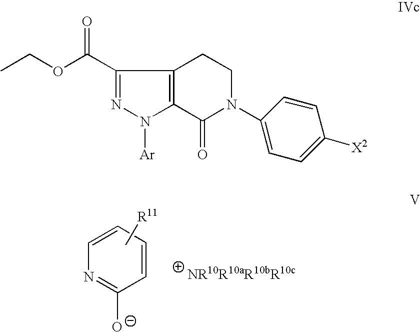
wherein:
X² is I;
Ar is selected from phenyl, 2-fluorophenyl, 3-chlorophenyl, and 4-methoxyphenyl;
R is selected from H, F, Cl, and OCH₃;
R¹⁰ is n-butyl;
R^{10a} is n-butyl;
R^{10c} is n-butyl;
R^{10c} is n-butyl; and
R¹¹ is H.

12. A process for preparing a compound of formula IIId, comprising:
(a) contacting a compound of formula Id with a compound of formula IId to form a compound of formula IIId; 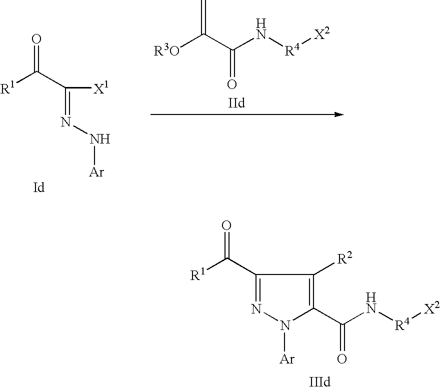
wherein:
X¹ is a leaving group selected from Cl, Br, and I;
X² is a leaving group selected from Cl, Br, I, OSO₂Me, OSO₂CF₃, OSO₂Ph, and OSO₂Ph-p-Me;
R¹ is selected from C₁₋₆ alkyl, C₀₋₆ alkylene-phenyl, O—C₁₋₆ alkyl, and O—C₀₋₆ alkylene-phenyl;
R³ is selected from C₁₋₆ alkyl, phenyl, and benzyl;
R⁴ is a 5-10 membered aromatic carbocyclic or heterocyclic ring consisting of carbon atoms and 0-4 heteroatoms selected from O, S(O)ₚ, and N and R⁴ is substituted with 0-2 groups selected from F and C₁₋₄ alkyl;
Ar is 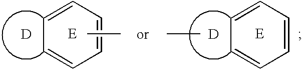
ring D, including the two atoms of Ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-2 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-2 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-1 carbonyl and 1-2 R and there are 0-3 ring double bonds;
R is selected from H, C₁₋₄ alkyl, F, Cl, Br, I, OH, OCH₃, OCH₂CH₃, OCH(CH₃)₂, OCH₂CH₂CH₃, (CR⁸R⁹)ₜC(O)R⁵, (CR⁸R⁹)ₜOR⁶, (CR⁸R⁹)ₜS(O)ₚR⁶, CN, C(═NR⁸)NR⁷R⁹, NHC(═NR⁸)NR⁷R⁹, ONHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜS(O)ₚNR⁷R⁸, (CR⁸R⁹)ₜNR⁷S(O)ₚR⁷, and OCF₃;
alternatively, when 2 R groups are attached to adjacent atoms, they combine to form methylenedioxy or ethylenedioxy;
R⁵, at each occurrence, is selected from CF₃, OH, C₁₋₄ alkoxy,
C₁₋₆ alkyl, —(CH₂)ᵣ—C₃₋₁₀ carbocycle substituted with 0-2 R^{5a}, and —(CH₂)ᵣ-5-10 membered heterocycle containing from 1-4 heteroatoms selected from the group consisting of N, O, and S(O)ₚ, and substituted with 0-2 R^{5a};
R^{5a}, at each occurrence, is selected from H, ═O, (CH₂)ᵣOR⁶, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR⁶R^{6a}, (CH₂)ᵣC(O)R⁶, (CH₂)ᵣC(O)OR⁶, (CH₂)ᵣNR⁶C(O)R⁶, (CH₂)ᵣ—C(O)NR⁶R^{6a}, (CH₂)ᵣNR⁶C(O)NR⁶R^{6a}, (CH₂)ᵣ—C(═NR⁶)NR⁶R^{6a}, (CH₂)ᵣNR⁶C(═NR⁶)NR⁶R^{6a}, (CH₂)ᵣSO₂NR⁶R^{6a}, (CH₂)ᵣNR⁶SO₂NR⁶R^{6a}, (CH₂)ᵣNR⁶SO₂—C₁₋₄ alkyl, (CH₂)ᵣNR⁶SO₂CF₃, (CH₂)ᵣNR⁶SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;
R⁶, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{6a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR⁶R^{6a} forms a 5 or 6 membered ring consisting of: carbon atoms, the nitrogen atom to which R⁶ and R^{6a} are attached, and 0-1 additional heteroatoms selected from the group consisting of N, O, and S(O)ₚ, and there are 0-3 ring double bonds;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₀₋₄ alkyl-C(O)—;
R⁸, at each occurrence, is selected from H, C₁₋₆ alkyl, and (CH₂)ₙ-phenyl;
alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
alternatively, R⁴—X² is selected from: 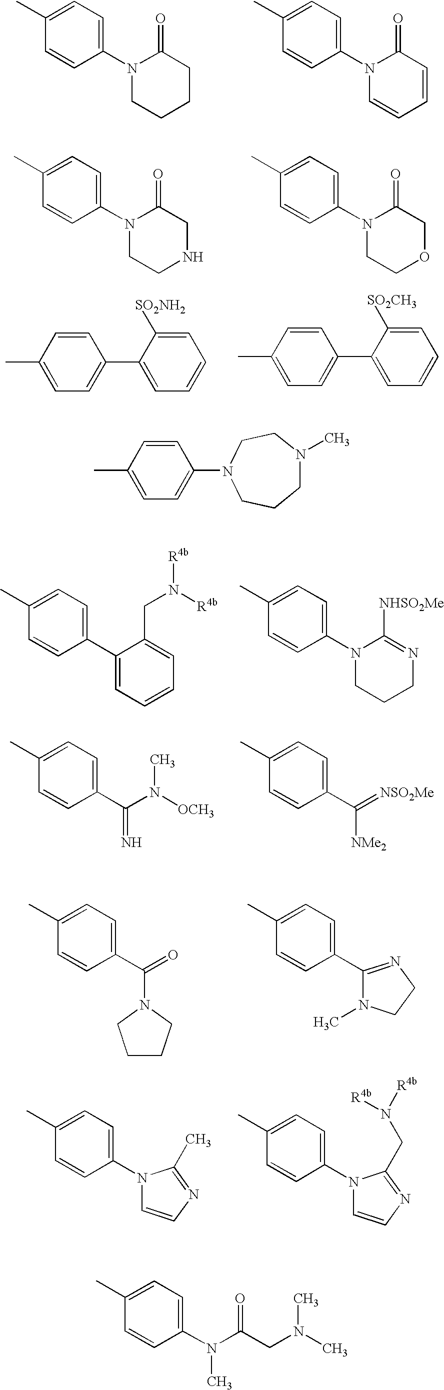 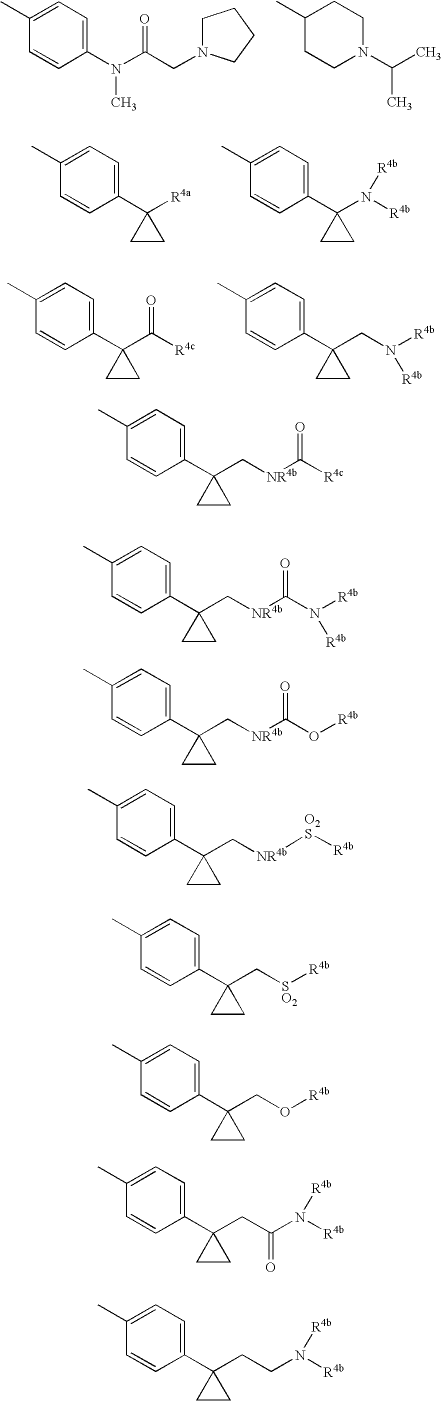 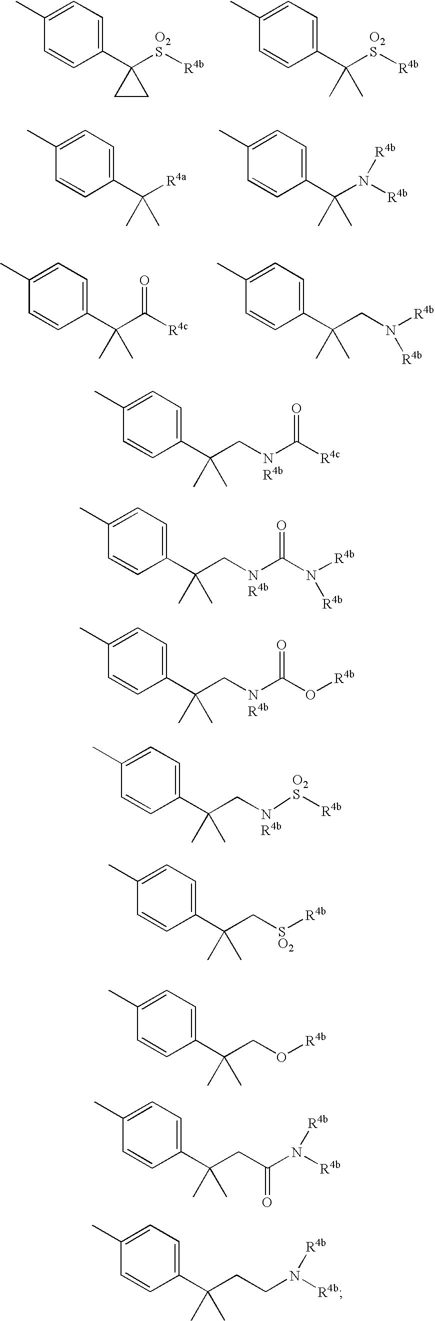
R^{4a} is substituted with 0-2 R^{4d} and selected from morpholine, 1,1-dioxo-thiomorpholine, dihydropyridine, piperidine, piperazine, pyrrolidine, imidazole, imidazoline, imidazolidine, oxazoline, and thiazoline;
R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;
R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;
R^{4d} is selected from —O, OH, OCH₃, and CH₃;
n, at each occurrence, is selected from 0, 1, 2, and 3;
p, at each occurrence, is selected from 0, 1, and 2;
r, at each occurrence, is selected from 0, 1, 2, 3, 4, 5, and 6; and
t, at each occurrence, is selected from 0, 1, 2, and 3.

13. A process according to claim 12, comprising:
(a₁) contacting a compound of formula Id with a compound of formula IId in the presence of a first base and a first solvent to form a cycloaddition product;
(a₂) contacting the cycloaddition product from reaction (a₁) with a first acid to form a compound of formula IIId;
wherein:
the first base is selected from a tertiary amine base and a pyridine base;
the first acid is selected from HCl, AcOH, H₂SO₄, and H₃PO₄; and
the first solvent is an aprotic solvent.

14. A process according to claim 12, the process, comprising: a process for preparing a compound of formula IIIe, comprising:
(a₁) contacting a compound of formula Ib with a compound of formula IIb in the presence of a first base and a first solvent to form a cycloaddition product;
(a₂) contacting the cycloaddition product from reaction (a₁) with a first acid to form a compound of formula IIIb; 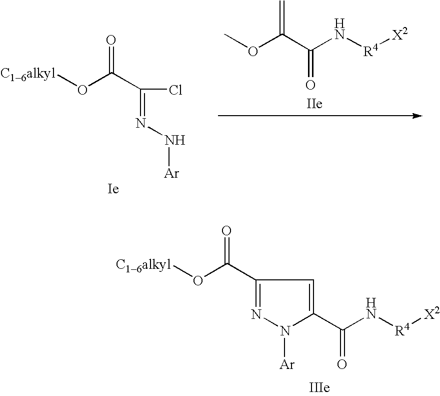
wherein:
the first base is triethylamine;
the first solvent is ethyl acetate
the first acid is HCl;
the second base is NaOEt;
the second solvent is EtOH;
X² is I;
R⁴ is selected from phenyl, pyridyl, and pyrimidyl;
Ar is selected from phenyl, 2-fluorophenyl, 3-aminomethyl-phenyl, 3-amidino-phenyl, 3-amido-phenyl, 3-chlorophenyl, 4-methoxyphenyl, 2-naphthyl, 1-fluoro-2-naphthyl, 1-cyano-2-naphthyl, and 6-chloro-2-naphthyl;
p, at each occurrence, is selected from 0, 1, and 2;
alternatively, R⁴—X² is selected from: 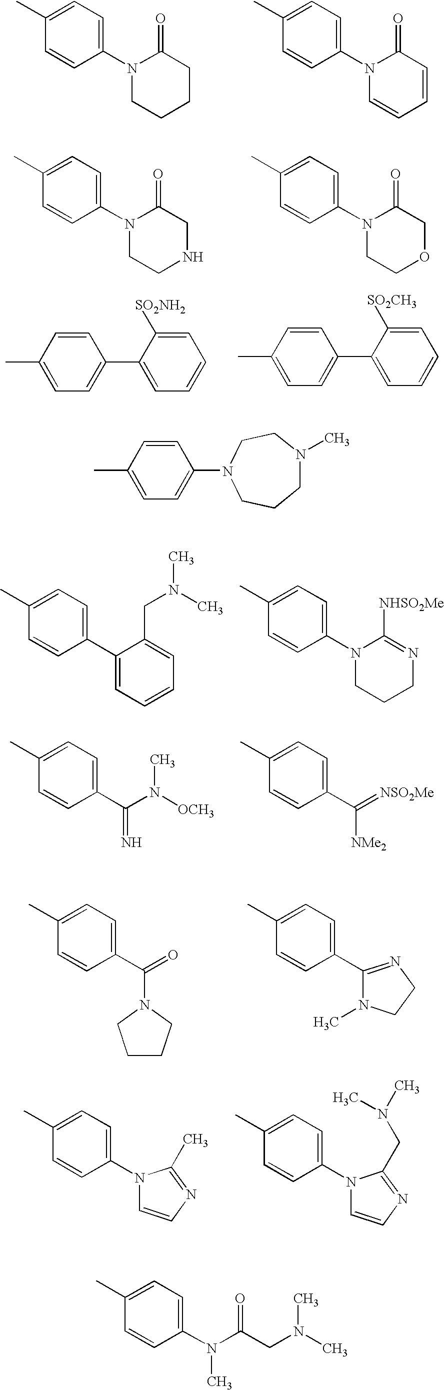 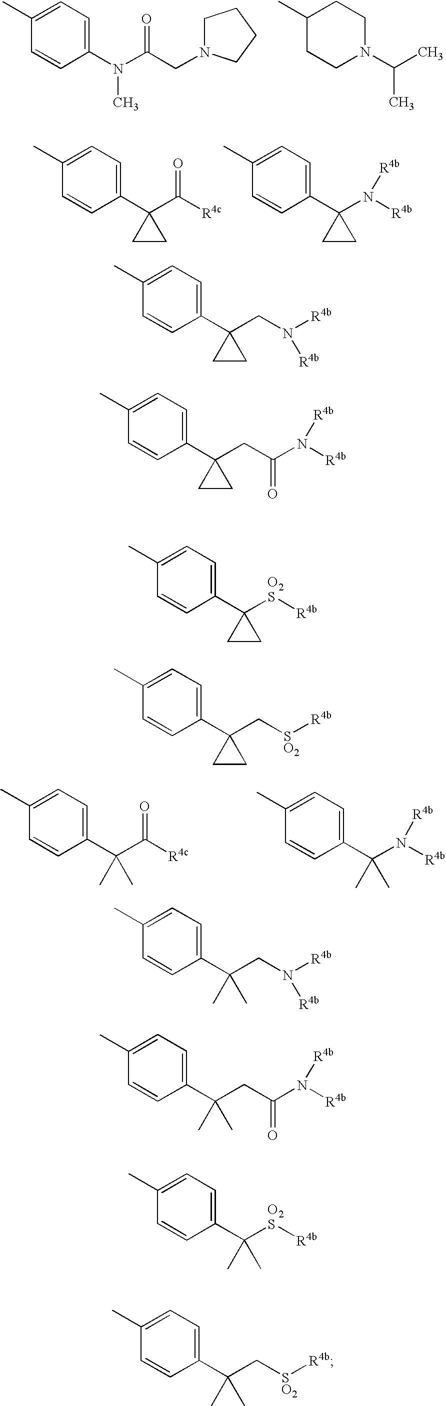
R^{4b}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;
R^{4c}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl; and
R^{4d} is selected from ═O, OH, OCH₃, and CH₃.
